(19) United States
(12) Patent Application Publication
(10) Pub. No.: US 2013/0331315 A1
(43) Pub. Date: Dec. 12, 2013

(54) Protein Hydrolysate Compositions Having Enhanced CCK and GLP-1 Releasing Activity

(71) Applicants: Elaine S. Krul, St. Louis, MO (US); Barry Tulk, St. Louis, MO (US); Mary K. Pawlik, St. Louis, MO (US); Jason F. Lombardi, St. Louis, MO (US)

(72) Inventors: Elaine S. Krul, St. Louis, MO (US); Barry Tulk, St. Louis, MO (US); Mary K. Pawlik, St. Louis, MO (US); Jason F. Lombardi, St. Louis, MO (US)

(73) Assignee: Solae, LLC, St. Louis, MO (US)

(21) Appl. No.: 14/001,292

(22) PCT Filed: Feb. 23, 2012
(86) PCT No.: PCT/US12/26250

§ 371 Date: Aug. 23, 2013

Related U.S. Application Data
(60) Provisional application No. 61/445,593, filed on Feb. 23, 2011.

(51) Int. Cl. C07K 14/415; A23L 1/305

(52) U.S. Cl. CPC C07K 14/415; A23L 1/3053
USPC 514/4.9; 530/350; 530/370; 530/378

(57) ABSTRACT

The present invention provides protein hydrolysate compositions having enhanced cholecystokinin (CCK) and/or giuoagon-like peptide-1 (GLP-1) releasing activity and food forms incorporating the protein hydrolysate compositions, which can be used to promote satiety.

### FIELD OF THE INVENTION

The present invention generally relates to protein hydrolysates. In particular, the protein hydrolysates of the invention have cholecystokinin (CCK) and/or glucagon-like peptide-1 (GLP-1) releasing activity. The protein hydrolysates can be used to provide nutrients and/or to promote satiety.

### BACKGROUND OF THE INVENTION

The number and rate of overweight and obese individuals and the diseases associated with increased body weight are rising in the Unites States and throughout the world. While there is no single underlying cause, a contributing factor may be the sedentary life styles of many individuals and the concomitant consumption of higher calorie foods, including “fast food.” Most “fast food” tends to be high in fat and/or sugar.

One viable target for combating the increased body weight epidemic may be CCK. CCK is a peptide hormone released into the circulation by gastrointestinal cells in response to nutrients, specifically protein or lipids consumed as a meal. CCK acts as a neurotransmitter, and neuromodulator in the central and the peripheral nervous systems. CCK is released from l enteroendocrine cells of the duodenum and jejunum in response to nutrients (e.g., protein and fat) that enter the gastrointestinal lumen after a meal. Once released, CCK initiates a number of responses coordinated to promote digestion and regulate food intake, including mediating bile emptying from the gall bladder, regulating the release of digestive enzymes from the pancreas, controlling gastric emptying by regulation of the pyloric sphincter, as well as neuronal signaling to the central nervous system via vagal afferent neurons. Neuronal CCK is believed to mediate a number of events within the CNS, including modulating dopaminergic neurotransmission and anxiogenic effects, as well as affecting cognition and nociception (see, e.g., J. N. Crawley and R. L. Corwin, 1994, Peptides, 15:731-755; N. S. Baber, C. T. Dourish, and D. R. Hill, Pain (1989), 39(3), 307-28; and P. De Tullio, J. Delarge and B. Pirotte, Expert Opinion on Investigational Drugs (2000), 9(1), 129-146). CCK has been shown to mediate its diverse hormonal and neuromodulatory functions through two receptor subtypes: the CCK-A (CCK-1) and CCK-B (CCK-2) subtypes (see, e.g., G. N. Woodruff and J. Hughes, Annu. Rev. Pharmacol. Toxicol. (1991), 31: 469-501). Both CCK-1 and CCK-2 receptor subtypes belong to the seven transmembrane G-protein-coupled superfamily of receptors. A number of studies suggest that CCK mediates its satiety effect through the CCK-1 receptor, which relays the postprandial satiety signal via the vagal afferents to the CNS inducing a “feeling” of satiety (see, e.g., G. P. Smith et al., Science 213 (1981) pp. 1036-1037; and J. N. Crawley et al., J. Pharmacol. Exp. Ther., 257 (1991) pp. 1076-1080).

CCK exerts some direct actions which induce satiety, including the inhibition of gastric emptying, inhibition of gastric acid secretion, and stimulation of gallbladder contraction. Whether through these direct effects on gastric emptying and intestinal digestion or through central nervous system pathways, CCK induces a sense of satiety which typically results in the consumption of fewer calories.

Another viable target for combating the increased body weight epidemic is GLP-1. GLP-1 has been described as an incretin hormone with a large array of effects. GLP-1 was discovered in 1984 and found to be an important incretin (Nauck, M. A.; Kleine, N.; Orskov, C.; Hoist, J. J.; Willms, B.; Creutzfeldt, W., Diabetologia 1993, 36, 741-744). GLP-1 is released by L cells in the distal ileum in response to glucose and fatty acids, however, it is known that peptides directly induce and/or modulate GLP-1 release (Hira T et al. (2009) Am J Physiol Gastrointest Liver Physiol 297: G663-G671). GLP-1 is released into the circulation following a meal and potently stimulates the release of insulin from the beta-cells in the pancreas in a glucose-dependent manner. Numerous additional effects have also been ascribed to GLP-1, including, stimulation of insulin biosynthesis, restoration of glucose sensitivity to the islets and stimulation of increased expression of the glucose transporter GLUT-2 and glucokinase. GLP-1 also has a number of effects on regulation of beta-cell mass, stimulation of replication and growth of existing beta-cells, inhibition of apoptosis and neogenesis of new beta-cells from duct precursor cells, which leads to reduced hepatic glucose output. Beneficial extrapancreatic effects of GLP-1 have also been reported such as a direct effect on reducing hepatic lipid content and improving cardiac function (Abu-Hamdah R. et al. J Clin Endocrinol Metab. 2009 June; 94(6):1843-52. Epub 2009 Mar. 31). In the gut, GLP-1 is a potent inhibitor of motility and gastric emptying and has also been shown to inhibit gastric acid secretion. The inhibition of gastric emptying leads to decreased food intake and reduced body weight over time (Flint, A.; Raben, A.; Astrup, A.; Hoist, J. J., J Clin Inv 1998, 101, 515-520; Zander, M.; Madsbad, S.; Madsen, J. L.; Hoist, J. J., Lancet 2002, 359, 824-830). GLP-1 has also been shown to have central effects on food intake through the action of GLP-1 receptors in the hypothalamic centers that control appetite (Barber T M et al. (2010) Maturitas doi:10.1016/j.maturitas.2010.06.018).

In light of the obesity epidemic and the dearth of effective means of combating it, there is a need for a nutritious, readily accessible ingredient or food product that can be consumed and that promotes weight loss or control. The food product should not only taste good, but it should also be nutritionally sound; that is, the product should be relatively low in fat, high in protein, and contain essential micronutrients (e.g. vitamins and minerals). In addition, it would also be highly beneficial if the ingredient or food product increased the release of CCK and/or GLP-1.

To this end, the present invention is drawn to novel protein hydrolysates, and food forms incorporating the same, that are able to promote weight management and satiety.

### SUMMARY OF INVENTION

The present invention provides protein hydrolysate compositions that are able to simulate CCK and/or GLP-1 releasing activity, and which can be used to promote weight management and satiety.

In certain embodiments, the invention is drawn to a protein hydrolysate composition comprising a mixture of polypeptide fragments, wherein the protein hydrolysate composition stimulates CCK and GLP-1 releasing activity.

In other certain embodiments, the invention is drawn to a food product containing a protein hydrolysate composition comprising a mixture of polypeptide fragments, wherein the protein hydrolysate composition stimulates CCK and GLP-1 releasing activity.

In other certain embodiments, the invention is drawn to a method of inducing satiety comprising ingesting a protein hydrolysate composition comprising a mixture of polypeptide fragments, wherein the protein hydrolysate composition stimulates CCK and GLP-1 releasing activity.

In other certain embodiments, the invention is drawn to a method of inducing satiety comprising ingesting a food product containing a protein hydrolysate composition comprising a mixture of polypeptide fragments, wherein the protein hydrolysate composition stimulates CCK and GLP-1 releasing activity.

### DESCRIPTION OF FIGURES

FIG. 1A. CCK release from STC-1 cells after incubation with undigested or digested intact proteins or protein hydrolysates.

FIG. 1B. GLP-1 release from STC-1 cells after incubation with undigested or digested intact proteins or protein hydrolysates.

FIG. 2. Illustrative chromatogram following size exclusion chromatography.

FIG. 3. Solubility of protein hydrolysate compositions over a pH range.

FIG. 4. Plot of the CCK and GLP-1 releasing activity of different soy protein hydrolysates created with various enzymes and treatment conditions described herein.

FIG. 5A. Dose response of CCK release by STC-1 cells after incubation with soy hydrolysates that also stimulate GLP-1 release.

FIG. 5B. Dose response of GLP-1 release by STC-1 cells after incubation with soy hydrolysates that also stimulate CCK release.

### DETAILED DESCRIPTION OF THE INVENTION

It is known that protein, in general, stimulates the release of CCK and glucose stimulates the release of GLP-1 by enteroendocrine cells in the intestinal tract of animals, including humans (Liddle, R. A., et al., (1986) Am. J. Physiol. Gastrointest. Liver Physiol. 251 (14): G243-G248; Jang H-J et al. (2007) PNAS 104 (38): 15069-15074). Soy protein hydrolysates have been shown to stimulate the release of CCK in the enteroendocrine cell model, STC-1 (PCT Application No. PCT/US2009/069867). Since protein is thought to modulate the release of GLP-1 (Hira T et al. (2009) Am J Physiol Gastrointest Liver Physiol 297: G663-G671) the effect of protein hydrolysates to induce GLP-1 release was investigated and is a subject of this invention.

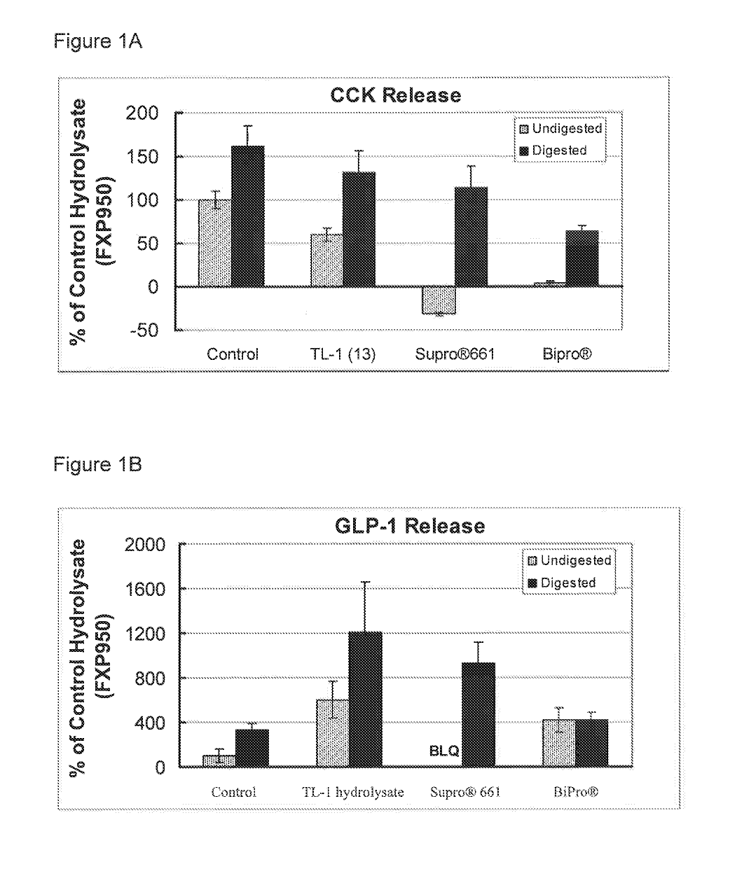

Protein that passes into the intestine after a meal experiences digestion by gastric enzymes such as pepsin at low pH then is further digested in the upper intestine by a mixture of pancreatic digestive enzymes (pancreatin). We show in FIG. 1 that a protein hydrolysate subjected to digestion by pepsin and pancreatin does not lose the ability to induce CCK and GLP-1 releasing activity on enteroendocrine cells. The soluble fraction of in vitro digested proteins or protein hydrolysates (2 mg/ml protein concentration) were incubated with STC-1 cells for 4 hours and 2 hours for the measurement of CCK and GLP-1, respectively. Cell culture media was harvested and assayed for CCK and GLP-1 by ELISA. The control soy protein hydrolysate was FXP950 (available commercially from Solae, LLC as SUPRO® FP 950). The intact proteins tested were soy protein isolate Supro® 661 and whey protein isolate BiPro®. The hydrolysed soy protein was generated by digestion with the enzyme TL-1 (trypsin-like protease (TL-1) from Fusarium oxysporum (SWISSPROT No. P35049) (U.S. Pat. Nos. 5,288,627 and 5,693,520 each of which is hereby incorporated by reference in its entirety)). The method for simulated digestion of the protein hydrolysates described herein used to generate the data is a modification of the previously published procedures of Schasteen and mimics in vivo gastrointestinal digestion (Schasteen, C. S., et al., (2007) Correlation of an Immobilized Digestive Enzyme Assay With Poultry True Amino Acid Digestibility for Soybean Meal. Poultry Science 86(2), 343-348) and Higaki ([Higaki, N., et al., (2006) Biosci. Biotechnol. Biochem. 70(12), 2844-2852). Protein samples were solubilized in 20 volumes of 0.01 M HCl and digested by pepsin (Sigma-Aldrich #P7012) at an enzyme-substrate ratio of 1:200 (w/w), pH 2.3 and 37° C. for 1 hour. After the pepsin digestion, 2.5 M NaOH was added to the mixture to adjust the pH to 8.0, and pancreatin (Sigma-Aldrich #P3292) was added at a ratio of 1:200 (w/w) and digestion was continued for another 4 hour. Degree of hydrolysis was determined by the reaction of primary amine groups in the pre-digested or digested samples with o-phthalaldehyde (OPA) vs. total amount of primary amine present in sample after acid hydrolysis (110° C. for 24 hrs) (known as the “OPA method”).

As taught and described herein, in vitro digested hydrolysates show significantly higher CCK and GLP-1 induction in STC-1 cells compared to an in vitro digested intact soy protein control. Digested samples were tested at five protein concentrations ranging from 0.07 to 6.0 mg/mL for CCK release and at concentrations ranging from 0.25 to 4.0 mg/mL for GLP-1 release. The higher level of CCK and GLP-1 release seen with the in vitro digested hydrolysates (to mimic in vivo digestion) compared to the in vitro digested intact soy protein suggests that in vivo the hydrolysates will induce more CCK and GLP-1 release by enteroendorcine cells in the gut that will result in increased satiety in an animal, including a human.

Additionally, protein hydrolysates of the invention demonstrate one or more advantages, including, for example, increased solubility relative to, for example, unhydrolyzed soy protein at acid pH (e.g., 3-4) and hedonic scores based on sensory data indicating that these would be suitable ingredients in a wide variety of food applications. Thus, the protein hydrolysates of the invention are promising novel ingredients that can be incorporated into a wide variety of foods, or used as stand-alones, that can be targeted to and used by individuals seeking foods with enhanced satiety effects to help manage food intake, preserve or increase lean body mass and/or maintain or lose non-lean body mass.

### I. Process for Preparing a Protein Hydrolysate

One aspect of the invention provides a protein hydrolysate and process for preparing the same. The process comprises contacting a protein material with one or more enzymes that cleave the protein material into polypeptide fragments that induce the release of CCK and/or GLP-1. In certain aspects of the invention, the polypeptide fragments induce the release of CCK. In other aspects of the invention, the polypeptide fragments induce the release of GLP-1. In further other aspects of the invention, the polypeptide fragments induce the release of CCK and GLP-1. Reactants and reaction parameters are described more fully below.

(a) Protein Material.

Non-limiting examples of suitable protein materials include plants, such as leguminous or non-leguminous plants (e.g., soybean and other legumes, channa (garbanzo), lentils; maize, peas, canola, sunflowers, sorghum, rice, amaranth, potato, tapioca, arrowroot, canna, lupin, rape, wheat, oats, rye, barley, buckwheat, cassava, triticale, millet, hemp, etc.), nuts and seeds (e.g., almonds, cashews, filberts, hemp seeds, peanuts, pumpkin seeds, sesame seeds, sunflower seeds, walnuts, etc.), animal proteins (e.g., egg proteins, dairy proteins, muscle proteins, gelatin, etc.), and combinations thereof.

In specific embodiments, the protein material is derived from soy. A variety of soy protein materials may be used in the process of the invention to generate a soy protein hydrolysate. In general, the soy protein material is derived from whole soybeans in accordance with methods known in the art. The whole soybeans may be standard soybeans (i.e., non-genetically modified soybeans), genetically modified soybeans (e.g., soybeans with modified oils, soybeans with modified carbohydrates, soybeans with modified protein subunits, and so forth) or combinations thereof. Suitable examples of soy protein material include soy extract, soymilk, soymilk powder, soy curd, defatted soy flour, partially defatted soy flour, full-fat soy flour, soy protein isolate, soy protein concentrate, soy whey protein, and fractions and mixtures thereof.

In one embodiment, the soy protein material used in the process is soy protein isolate (also called isolated soy protein or ISP). In general, a soy protein isolate has a protein content of at least about 90% soy protein on a moisture-free basis. The soy protein isolate may comprise intact soy proteins or it may comprise partially hydrolyzed soy proteins. The soy protein isolate may have a high content of various subunits such as 7S, 11S, 2S, etc. Non-limiting examples of a soy protein isolate that may be used in the present invention are commercially available, for example, from Solae, LLC (St. Louis, Mo.), and include SUPRO® 500E, SUPRO® 620, SUPRO® 760, SUPRO® 670, SUPRO® 710, SUPRO® EX 33, SUPRO® 313.

In another embodiment, the soy protein material is soy protein concentrate, which has a protein content of about 65% to less than about 90% soy protein on a moisture-free basis. Examples of suitable soy protein concentrates useful in the invention include, for example, ALPHA® DSP-C, Procon™, ALPHA® 12 and ALPHA® 5800, which are commercially available from Solae, LLC. Alternatively, a soy protein concentrate may be blended with a soy protein isolate to substitute for a portion of the soy protein isolate as a source of protein material.

In another embodiment, the soy protein material is soy flour, which has a protein content of about 49% to about 65% soy protein on a moisture-free basis. The soy flour may be defatted soy flour, partially defatted soy flour, or full-fat soy flour. The soy flour may be blended with a soy protein isolate or a soy protein concentrate.

When soy flour is used, the starting material is typically defatted soy flour or flakes. Full-fat soybeans contain approximately 40% protein by weight and approximately 20% oil by weight. Whole full-fat soybeans may be defatted through conventional processes when defatted soy flour or flakes form the starting protein material. For example, the soybean may be cleaned, dehulled, cracked, passed through a series of flaking rolls and then subjected to solvent extraction by use of hexane, or other appropriate solvent, to extract the oil and produce “spent flakes.” The defatted flakes may be ground to produce soy flour. Although the process is yet to be employed with full-fat soy flour, it is believed that full-fat soy flour may also serve as a protein source. However, where full-fat soy flour is processed, it is most likely necessary to use a separation step, such as three stage centrifugation to remove oil.

In another embodiment, the soy protein material is one or more soy storage proteins that have been separated into major fractions (15S, 11 S, 7S, and 2S) on the basis of, for example, sedimentation in a centrifuge. In general, the 11S fraction is highly enriched in glycinins, and the 7S fraction is highly enriched in beta-conglycinins.

In another embodiment, the protein material may be derived from a plant other than soy. By way of non-limiting example, suitable plants include other legumes, channa (garbanzo), lentils, maize, peas, canola, sunflowers, sorghum, rice, amaranth, potato, tapioca, arrowroot, canna, lupin, rape, wheat, oats, rye, barley, buckwheat, cassava, triticale, millet, hemp, and mixtures thereof. In specific embodiments, the plant protein material is canola meal, canola protein isolate, canola protein concentrate, or combinations thereof. In another embodiment, the plant protein material is maize or corn protein powder, maize or corn protein concentrate, maize or corn protein isolate, maize or corn germ, maize or corn gluten, maize or corn gluten meal, maize or corn flour, zein protein, or combinations thereof. In still another embodiment, the plant protein material is barley powder, barley protein concentrate, barley protein isolate, barley meal, barley flour, or combinations thereof. In an alternate embodiment, the plant protein material is lupin flour, lupin protein isolate, lupin protein concentrate, or combinations thereof. In another alternate embodiment, the plant protein material is oatmeal, oat flour, oat protein flour, oat protein isolate, oat protein concentrate, or combinations thereof. In yet another embodiment, the plant protein material is pea flour, pea protein isolate, pea protein concentrate, or combinations thereof. In still another embodiment, the plant protein material is potato protein powder, potato protein isolate, potato protein concentrate, potato flour, or combinations thereof. In a further embodiment, the plant protein material is rice flour, rice meal, rice protein powder, rice protein isolate, rice protein concentrate, or combinations thereof. In another alternate embodiment, the plant protein material is wheat protein powder, wheat gluten, wheat germ, wheat flour, wheat protein isolate, wheat protein concentrate, solubilized wheat proteins, or combinations thereof.

In other embodiments, the protein material is derived from an animal source. In one embodiment, the animal protein material is derived from eggs. Non-limiting examples of suitable egg proteins include powdered egg, dried egg solids, dried egg white protein, liquid egg white protein, egg white protein powder, isolated ovalbumin protein, and combinations thereof. Egg proteins may be derived from, for example, eggs of chicken, duck, goose, quail, or other birds. In an alternate embodiment, the protein material is derived from a dairy source. Suitable dairy proteins include, for example, non-fat dry milk powder, milk protein isolate, milk protein concentrate, acid casein, caseinate (e.g., sodium caseinate, calcium caseinate, etc.), whey protein isolate, whey protein concentrate, and combinations thereof. The milk protein material may be derived from, for example, cows, goats, sheep, donkeys, camels, camelids, yaks, water buffalos, etc. In a further embodiment, the protein is derived from the muscles, organs, connective tissues, or skeletons of land-based or aquatic animals. In certain embodiments, the animal protein is gelatin, which is produced by partial hydrolysis of collagen extracted from the bones, connective tissues, organs, etc. from cattle or other animals.

It is also envisioned that combinations of a soy protein material and at least one other protein material also may be used in the invention. That is, a protein hydrolysate composition may be prepared from a combination of a soy protein material and at least one other protein material. In one embodiment, a protein hydrolysate composition is prepared from a combination of a soy protein material and one other protein material. In another embodiment, a protein hydrolysate composition is prepared from a combination of a soy protein material and two other protein materials. In further embodiments, a protein hydrolysate composition is prepared from a combination of a soy protein material and three or more other protein materials.

In other embodiments, a protein hydrolysate composition further comprises at least one nonhydrolyzed protein from a protein material. Non-limiting examples of suitable nonhydrolyzed proteins include dry milk powder, non-fat dry milk powder, milk proteins, acid casein, caseinate (e.g., sodium caseinate, calcium caseinate, etc.), whey protein concentrate, whey protein isolate, and soy protein isolate.

The concentration of protein from the soy protein material and protein from the other protein material used in combination can and will vary. The amount of protein from the soy protein material may range from about 1% to about 99% of the total protein used in the combination. In certain embodiments, the amount of protein from the soy protein material ranges from about 1% to about 99%, about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% of the total protein used in the combination. Likewise, the amount of the (at least one) other protein material may range from about 1% to about 99%, about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% of the total protein used in the combination.

(b) Protein Slurry.

In the process of the invention, the protein material is typically mixed or dispersed in water to form a slurry comprising about 1% to about 40% protein by weight (on an as-is basis). In certain embodiments, the slurry may comprise about 1% to about 40% protein, about 5% to about 35% protein, about 10% to about 25% protein, or about 15% to about 20% protein (as-is) by weight. In a specific embodiment, the slurry may comprise less than about 10% protein (as-is) by weight. In a further specific embodiment, the slurry may comprise about 2%, about 4%, about 6%, about 8%, or about 10% protein (as-is) by weight. The water may include food grade dispersants such as ethanol, glycerol, and the like.

After the protein material is dispersed in water, the slurry of protein material may be heated from about 70° C. to about 90° C. for about 2 minutes to about 20 minutes (or at a higher temperature from about 110° C. to about 177° C. for about 2 seconds to about 30 seconds) to inactivate putative endogenous protease inhibitors. Typically, the pH and the temperature of the protein slurry are adjusted so as to optimize the hydrolysis reaction, and in particular, to ensure the digestion enzyme used in the hydrolysis reaction functions near its optimal activity level. The pH of the protein slurry may be adjusted and monitored according to methods generally known in the art. The pH of the protein slurry may be adjusted and maintained at from about 2.0 to about 11.0. In other embodiments, the pH of the protein slurry may be adjusted and maintained at from about 2.0 to about 3.0, about 3.0 to about 4.0, about 4.0 to about 5.0, about 5.0 to about 6.0, about 6.0 to about 7.0, about 7.0 to about 8.0, about 8.0 to about 9.0, about 9.0 to about 10.0, or about 10.0 to about 11.0. In another embodiment, the pH of the protein slurry may be adjusted and maintained at from about 6.0 to about 9.0. In another embodiment, the pH of the protein slurry may be adjusted and maintained at from about 7.0 to about 8.0. The temperature of the protein slurry can be adjusted and maintained at from about 25° C. to about 80° C. during the hydrolysis reaction in accordance with methods known in the art. In certain embodiments, the temperature of the protein slurry can be adjusted and maintained at about 50° C. during the hydrolysis reaction in accordance with methods known in the art. The temperature should not reach a point where a significant amount of the hydrolysis enzyme is inactivated (e.g., by denaturation).

(c) Enzyme Digestion.

The hydrolysis reaction is generally initiated by adding an enzyme or a combination of enzymes to the slurry of protein material. Typically, the enzyme may be a food-grade enzyme having optimal activity at a pH from about 2.0 to about 11.0 and at a temperature from about 25° C. to about 80° C. The enzyme may be of plant, animal, or microbial origin.

In certain embodiments of the invention, the enzyme is an endopeptidase. Endopeptidases act preferentially in the inner regions of peptide chains away from the N and C termini. Several endopeptidases are suitable for use to practice the invention. In one embodiment, the peptidase is TL-1. In another embodiment, the peptidase is a bacterial protease from Bacillus amyloliquefaciens sold under the name NEUTRASE®.

In other embodiments of the invention, the endopeptidase is serine protease from Nocardiopsis prasina (SEQ ID NO: 2 in International Application No. WO 2005035747). In another embodiment, the endopeptidase is subtilisin protease from Bacillus licheniformis, which is available as ALCALASE® from Novozymes (Bagsvaerd, Denmark). In yet another embodiment, the endopeptidase is serine protease also called glutamyl endopeptidase (termed “GE”) from Bacillus licheniformis (UNIPROT: P80057 as disclosed and characterized in U.S. Pat. Nos. 4,266,031, 5,874,278, and 5,459,064 and International Application Nos. WO 01/16285, WO 92/13964, WO 91/13553, and WO 91/13554, each of which is incorporated by reference in its entirety). In an alternate embodiment, the endopeptidase is lysyl endopeptidase (termed “LE”) from Achromobacter lyticus (UNIPROT:P15636). In a further embodiment, the endopeptidase is a more purified form of subtilisin protease from Bacillus licheniformis (termed “Alcalase®”). In still other embodiments, the endopeptidase is a trypsin-like protease from Fusarium solani (GENESEQP:ADZ80577). Suitable enzymes further include, for example, SP 1 (a protease derived from Nocardiopsis sp. NRRL 18262 disclosed in WO 2001/058276 and WO 2009/155557), subtilisin protease 2 (S2), metallo protease 1 (MP1), and aspartate protease 1 (ASP-1). Other suitable enzymes include, for example bromelain, subtilisin, chymotrypsin, trypsin, pepsin, and elastase. In other embodiments of the invention, a combination of endopeptidases is used.

In a further embodiment of the invention, the endopeptidase is combined with at least one exopeptidase. Generally, exopeptidases act only near the ends of polypeptide chains at the N or C terminus. Those acting at a free N terminus liberate, for example, a single amino acid residue (i.e., aminopeptidases), a dipeptide (i.e., dipeptidyl-peptidases) or a tripeptide (i.e., tripeptidyl-peptidases). The exopeptidases acting at a free C terminus liberate, for example, a single amino acid (i.e., carboxypeptidases) or a dipeptide (i.e., peptidyl-dipeptidases). Some exopeptidases are specific for dipeptides (i.e., dipeptidases) or remove terminal residues that are substituted, cyclized or linked by isopeptide bonds. Isopeptide bonds are peptide linkages other than those of a carboxyl group to an α-amino group, and this group of enzymes is characterized by omega peptidases.

Non-limiting examples of exopeptidases include, for example, carboxypeptidase D from Aspergillus oryzae (UNIPROT:Q2TZ11), carboxypeptidase Y from Aspergillus oryzae (UNIPROT: Q2TYA1), aminopeptidase from Aspergillus oryzae (International Application No. WO 96/28542, which is incorporated by reference in its entirety), and aminopeptidase from Bacillus licheniformis (UNIPROT:Q65DH7.

The amount of enzyme added to the protein material can and will vary, depending upon the desired degree of hydrolysis and the duration of the hydrolysis reaction. The amount may range from about 1 mg to about 5000 mg of enzyme protein per kilogram of solids. In certain embodiments of the invention, the amount of enzyme ranges from about 1 mg to about 1000 mg of enzyme protein per kilogram solids, about 1 mg to about 900 mg of enzyme protein per kilogram, solids, about 1 mg to about 800 mg of enzyme protein per kilogram solids, about 1 mg to about 700 mg of enzyme protein per kilogram solids, about 1 mg to about 600 mg of enzyme protein per kilogram solids, about 1 mg to about 500 mg of enzyme protein per kilogram solids, about 1 mg to about 400 mg of enzyme protein per kilogram solids, and about 1 mg to about 300 mg of enzyme protein per kilogram solids.

In other certain embodiments, the amount of enzyme ranges from about 1 mg to about 250 mg of enzyme protein per kilogram solids. In yet even another embodiment, the amount of enzyme ranges from about 1 mg to about 200 mg of enzyme protein per kilogram solids. In yet even another embodiment, the amount of enzyme ranges from about 1 mg to about 100 mg of enzyme protein per kilogram solids. In yet even another embodiment, the amount of enzyme ranges from about 1 mg to about 50 mg of enzyme protein per kilogram solids. In yet even another embodiment, the amount of enzyme ranges from about 1 mg to about 25 mg of enzyme protein per kilogram solids. In yet even another embodiment, the amount of enzyme ranges from about 1 mg to about 10 mg of enzyme protein per kilogram solids. In specific embodiments, the amount of enzyme ranges from about 75 mg to about 150 mg of enzyme protein per kilogram solids. In other specific embodiments, the amount of enzyme is about 75 mg of enzyme protein per kilogram solids. In further other specific embodiments, the amount of enzyme is about 100 mg of enzyme protein per kilogram solids. In yet further other specific embodiments, the amount of enzyme is about 150 mg of enzyme protein per kilogram solids.

As will be appreciated by a skilled artisan, the duration of the hydrolysis reaction can and will vary depending upon the enzyme, the protein material, and the desired degree of hydrolysis. Generally speaking, the duration of the hydrolysis reaction may range from a few minutes to many hours, such as, from about 30 minutes to about 48 hours. To end the hydrolysis reaction, the composition may be heated to a temperature that is high enough to inactivate the enzyme. For example, heating the composition to a temperature of approximately 90° C. will substantially heat-inactivate most enzymes. Other methods of inactivation include cooling below 10° C. and/or lowering pH below about 2.0, depending on the enzyme used.

### II. Protein Hydrolysate

The protein hydrolysate compositions of the invention generally enhance CCK and GLP-1 release and thereby promote satiety when consumed. In certain embodiments, a protein hydrolysate composition of the invention contains the soluble fraction, insoluble fraction, or combinations thereof. In other embodiments, a protein hydrolysate composition of the invention has one or more of the properties described herein (including, for example, the enzyme used, DH, potency of CCK releasing activity, potency of GLP-1 releasing activity, MW distribution, solubility, or other characteristics).

As illustrated in the examples, a protein hydrolysate composition of the invention stimulates CCK and/or GLP-1 releasing activity. In particular, a protein hydrolysate composition of the invention subjected to digestion by pepsin and pancreatin does not lose the ability to induce CCK and GLP-1 releasing activity on enteroendocrine cells. The higher level of CCK and GLP-1 release seen with the in vitro digested hydrolysates (to mimic in vivo digestion) compared to the in vitro digested intact soy protein suggests that in vivo the hydrolysates will induce more CCK and GLP-1 release by enteroendorcine cells in the gut that will result in increased satiety in an animal, including a human.

In certain embodiments of the invention, a protein hydrolysate composition described herein stimulates CCK releasing activity. In other certain embodiments, a protein hydrolysate composition described herein stimulates GLP-1 releasing activity. In further other certain embodiments, a protein hydrolysate composition described herein stimulates CCK and GLP-1 releasing activity. In particular embodiments, a protein hydrolysate composition of the invention is digested (i.e., pepsin-pancreatin digested), undigested (i.e., no pepsin-pancreatin digested), or combinations thereof.

In one embodiment of the invention, a protein hydrolysate composition of the invention stimulates CCK releasing activity from about 50% to about 1000% of CCK released by STC-1 cells stimulated with 2 mg/ml FXP950 for 4 hours. In another embodiment, a protein hydrolysate composition of the invention stimulates CCK releasing activity from about 50% to about 500% of CCK released by STC-1 cells stimulated with 2 mg/ml FXP950 for 4 hours. In another embodiment, a protein hydrolysate composition of the invention stimulates CCK releasing activity from about 50% to about 400% of CCK released by STC-1 cells stimulated with 2 mg/ml FXP950 for 4 hours. In another embodiment, a protein hydrolysate composition of the invention stimulates CCK releasing activity from about 50% to about 300% of CCK released by STC-1 cells stimulated with 2 mg/ml FXP950 for 4 hours. In another embodiment, a protein hydrolysate composition of the invention stimulates CCK releasing activity from about 50% to about 200% of CCK released by STC-1 cells stimulated with 2 mg/ml FXP950 for 4 hours. In another embodiment, a protein hydrolysate composition of the invention stimulates CCK releasing activity from about 50% to about 100% of CCK released by STC-1 cells stimulated with 2 mg/ml FXP950 for 4 hours.

In particular embodiments, a protein hydrolysate composition of the invention stimulates CCK releasing activity from about 50%, about 60%, about 70%, about 80%, about 90%, about 100%, about 110%, about 120%, about 130%, about 140%, about 150%, about 160%, about 170%, about 180%, about 190%, or about 200% of CCK released by STC-1 cells stimulated with 2 mg/ml FXP950 for 4 hours.

In another embodiment of the invention, a protein hydrolysate composition of the invention stimulates GLP-1 releasing activity from about 50% to about 1000% of GLP-1 released by STC-1 cells stimulated with 2 mg/ml FXP950 for 2 hours. In another embodiment, a protein hydrolysate composition of the invention stimulates GLP-1 releasing activity from about 50% to about 500% of GLP-1 released by STC-1 cells stimulated with 2 mg/ml FXP950 for 2 hours. In another embodiment, a protein hydrolysate composition of the invention stimulates GLP-1 releasing activity from about 50% to about 400% of GLP-1 released by STC-1 cells stimulated with 2 mg/ml FXP950 for 2 hours. In another embodiment, a protein hydrolysate composition of the invention stimulates GLP-1 releasing activity from about 50% to about 300% of GLP-1 released by STC-1 cells stimulated with 2 mg/ml FXP950 for 2 hours. In another embodiment, a protein hydrolysate composition of the invention stimulates GLP-1 releasing activity from about 50% to about 200% of GLP-1 released by STC-1 cells stimulated with 2 mg/ml FXP950 for 2 hours. In another embodiment, a protein hydrolysate composition of the invention stimulates GLP-1 releasing activity from about 50% to about 100% of GLP-1 released by STC-1 cells stimulated with 2 mg/ml FXP950 for 2 hours.

In particular embodiments, a protein hydrolysate composition of the invention stimulates GLP-1 releasing activity from about 50%, about 60%, about 70%, about 80%, about 90%, about 100%, about 110%, about 120%, about 130%, about 140%, about 150%, about 160%, about 170%, about 180%, about 190%, or about 200% of GLP-1 released by STC-1 cells stimulated with 2 mg/ml FXP950 for 2 hours.

The degree of hydrolysis (DH) of the protein hydrolysate composition can and will vary depending upon the source of the protein material, the protease(s) used, and the conditions of the hydrolysis reaction. DH refers to the percentage of peptide bonds cleaved versus the starting number of peptide bonds. For example, if a starting protein containing five hundred peptide bonds is hydrolyzed until fifty of the peptide bonds are cleaved, then the DH of the resulting hydrolysate is 10%. DH may be determined using the trinitrobenzene sulfonic (TNBS) method or the ortho-phthaldialdehye (OPA) method, which are known to those skilled in the art. The higher the DH the greater the extent of protein hydrolysis. Typically, as the protein is further hydrolyzed (i.e., the higher the DH), the molecular weight of the peptide fragments decreases and the peptide profile changes accordingly. DH may be measured in the entire hydrolysate (i.e., whole fraction) or the DH may be measured in certain fractions of the hydrolysate (e.g., a soluble fraction, a molecular weight fraction, etc.).

Typically, each of the protein hydrolysate compositions of the invention will have a degree of hydrolysis that ranges from about 0.01% to about 35%. In one embodiment, the DH of a protein hydrolysate composition of the invention ranges from about 0.01% to about 20%. In another embodiment, the OH of a protein hydrolysate composition of the invention ranges from about 0.01% to about 10%. In another embodiment, the DH of a protein hydrolysate composition of the invention ranges from about 0.05% to about 10%. In specific embodiments, the DH of a protein hydrolysate composition of the invention ranges from about 0.01%, about 0.05%, about 0.1%, about 0.2%, about 0.3%, about 0.4% about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0%, about 10.5%, about 11.0%, about 11.5%, about 12.0%, about 12.5%, about 13.0%, about 13.5%, about 14.0%, about 14.5%, about 15.0%, about 15.5%, about 16.0%, about 16.5%, about 17.0%, about 17.5%, about 18.0%, about 18.5%, about 19.0%, about 19.5%, about 20.0%, about 20.5%, about 21.0%, about 21.5%, about 22.0%, about 22.5%, about 23.0%, about 23.5%, about 24.0%, about 24.5%, about 25.0%, about 25.5%, about 26.0%, about 26.5%, about 27.0%, about 27.5%, about 28.0%, about 28.5%, about 29.0%, about 29.5%, about 30.0%, about 30.5%, about 31.0%, about 31.5%, about 32.0%, about 32.5%, about 33.0%, about 33.5%, about 34.0%, about 34.5%, or about 35.0%.

In general, a protein hydrolysate composition of the invention, compared with the protein starting material, will comprise a mixture of polypeptide fragments of varying lengths and molecular weights. The molecular weight of the peptide fragments may range from 75 Daltons (i.e., free glycine) to greater than 100,000 Daltons, as measured by, for example, size exclusion chromatography. In general, the polypeptide fragments of the protein hydrolysate compositions of the invention have fractions of polypeptide fragments of varying lengths and molecular weights. A representative sample of each of the hydrolysates were prepared as described in the Examples and resuspended in phosphate-buffered saline (PBS, pH 7.4, Sigma cat#P5368) at a solids concentration of 2.5%. Insoluble material was removed by centrifugation at 16,000×g for 10 minutes, and the resulting supernatant passed through a 0.45 micron syringe filter to remove any remaining fines.

In one embodiment, about 30% to about 50% of polypeptides in the soluble fraction of a protein hydrolysate composition of the invention have a molecular weight greater than about 20 kDa. In specific embodiments, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of polypeptides in the soluble fraction of a protein hydrolysate composition of the invention have a molecular weight greater than about 20 kDa.

In another embodiment, about 15% to about 20% of polypeptides in the soluble fraction of a protein hydrolysate composition of the invention have a molecular weight between about 10 kDa and about 20 kDa. In specific embodiments, about 15%, about 16%, about 17%, about 18%, about 19%, or about 20% of polypeptides in the soluble fraction of a protein hydrolysate composition of the invention have a molecular weight between about 10 kDa and about 20 kDa.

In another embodiment, about 15% to about 20% of polypeptides in the soluble fraction of a protein hydrolysate composition of the invention have a molecular weight between about 5 kDa and about 10 kDa. In specific embodiments, about 15%, about 16%, about 17%, about 18%, about 19%, or about 20% of polypeptides in the soluble fraction of a protein hydrolysate composition of the invention have a molecular weight between about 5 kDa and about 10 kDa.

In another embodiment, about 15% to about 20% of polypeptides in the soluble fraction of a protein hydrolysate composition of the invention have a molecular weight between about 2 kDa and about 5 kDa. In specific embodiments, about 15%, about 16%, about 17%, about 18%, about 19%, or about 20% of polypeptides in the soluble fraction of a protein hydrolysate composition of the invention have a molecular weight between about 2 kDa and about 5 kDa.

In another embodiment, about 5% to about 10% of polypeptides in the soluble fraction of a protein hydrolysate composition of the invention have a molecular weight between about 1 kDa and about 2 kDa. In specific embodiments, about 5%, about 6%, about 7%, about 8%, about 9%, or about 10% of polypeptides in the soluble fraction of a protein hydrolysate composition of the invention have a molecular weight between about 1 kDa and about 2 kDa.

In another embodiment, less than about 5% of polypeptides in the soluble fraction of a protein hydrolysate composition of the invention have a molecular weight of less than about 1 kDa. In specific embodiments, less than about 1%, less than about 2%, less than about 3%, less than about 4%, or less than about 5% of polypeptides in the soluble fraction of a protein hydrolysate composition of the invention have a molecular weight of less than about 1 kDa.

In general, a protein hydrolysate composition of the invention has increased solubility compared to the protein starting material. The solubility will differ over a range of pHs.

In certain embodiments of the invention, a protein hydrolysate composition of the invention is soluble between about pH 3 and about pH 8.

In other certain embodiments, a protein hydrolysate composition of the invention is about 10% to about 60% soluble between about pH 3 and about pH 5. In specific embodiments, the solubility over this pH range is about 30% to about 60%, about 35% to about 55%, or about 40% to about 50%. In other specific embodiments, the solubility over this pH range is about 10%, about 15%, about 20%, about 25%, about 30%, about 35%, about 40%, about 45%, about 50%, about 55%, or about 60%. In further other specific embodiments, the pH over this pH range is about pH 3.0, about pH 3.25, about pH 3.5, about pH 3.75, about pH 4.0, about pH 4.25, about pH 4.5, about pH 4.75, or about pH 5.0. In yet further other specific embodiments, the pH over this range is about pH 3 to about pH 4.

In further certain embodiments, a protein hydrolysate composition of the invention is about 20% to about 75% soluble between about pH 5 and about pH 6. In specific embodiments, the solubility over this pH range is about 20%, about 25%, about 30%, about 35%, about 40%, about 45%, about 50%, about 55%, about 60%, about 65%, about 70%, or about 75%. In other specific embodiments, the pH over this pH range is about pH 5.0, about pH 5.25, about pH 5.5, about pH 5.75, or about pH 6.0.

In further certain embodiments, a protein hydrolysate composition of the invention is about 40% to about 85% soluble between about pH 6 and about pH 8. In specific embodiments, the solubility over this pH range is about 55% to about 85%, about 60% to about 80%, or about 65% to about 75%. In other specific embodiments, the solubility over this pH range is about 40%, about 45%, about 50%, about 55%, about 60%, about 65%, about 70%, about 75%, about 80%, or about 85%. In further other specific embodiments, the pH over this pH range is about pH 6.0, about pH 6.25, about pH 6.5, about pH 6.75, about pH 7.0, about pH 7.25, about pH 7.5, about pH 7.75, or about pH 8.0.

In other embodiments, a protein hydrolysate composition of the invention has a blood cholesterol lowering effect that enables an FDA approved heart health claim to be made, provided all the requisite requirements in the end food form are met (e.g., quantity of protein per serving, quantity of saturated fat per serving, etc.). Determining blood cholesterol lowering effects are known in the art and, thus, can be determined for a protein hydrolysate composition of the invention or food product incorporating the same.

### III. Food Products Comprising a Protein Hydrolysate

A further aspect of the present invention is a food product comprising an edible material and a protein hydrolysate composition described herein.

The selection of a particular protein hydrolysate composition to combine with an edible material can and will vary depending upon the desired food product.

In some embodiments, the protein hydrolysate composition included in the food product comprises “pre-peptides” that are converted into “active” peptides via proteolytic digestion in the stomach and/or intestine of the subject. In other embodiments, the protein hydrolysate composition comprises “active” peptides that require no additional proteolytic digestion in the stomach or intestines of the subject.

In some embodiments, a protein hydrolysate composition of the invention included in the desired food product is the whole unfractionated composition, which includes, for example, all of the soluble and insoluble fractions. For example, a whole unfractionated protein hydrolysate composition of the invention may be used in baked goods. In other embodiments, a protein hydrolysate composition of the invention included in the desired food product is a fractionated composition, which includes, for example, soluble or insoluble fractions. For example with regard to the soluble fraction, the soluble fraction of a protein hydrolysate composition of the invention can be used in acid beverages. The soluble fraction can be determined and incorporated in the desired food product by one of ordinary skill in the art by, for example, adjusting the pH to the desired level and removing any remaining insoluble material by methods know in the art including, for example, centrifugation, membrane fractionation, and combinations thereof. For example with regard to the insoluble fraction, the insoluble fraction of a protein hydrolysate composition of the invention can be used in cereals.

In certain embodiments of the invention, the insoluble fraction of a protein hydrolysate composition of the invention is important. The inventors have found that in addition to the ability of a soluble fraction of a protein hydrolysate composition of the invention to induce CCK and GLP-1 release, insoluble fractions also have these biological activities. As such, soluble fractions, insoluble fractions, or combinations thereof are encompassed by the invention as inducing CCK and GLP-1 release and use in a food product comprising an edible material and a protein hydrolysate composition described herein.

The selection of the appropriate edible material also will vary depending on the desired food product. The edible material may be a plant-derived material (e.g., a vegetable juice, a cereal product, etc.), an animal-derived material (e.g., a dairy product, an egg product, etc.), or a biomaterial (e.g., a protein, a carbohydrate, a lipid, etc.) isolated from a plant-derived material or an animal-derived material, and so forth.

A food product of the invention may include, for example, hot or cold cereals, bars, baked goods, beverages, yogurts, desserts, snacks, pastas, and meats (including poultry and seafood).

In one embodiment, the food product may be a liquid beverage. Non-limiting examples of liquid beverages include fruit juices, fruit drinks, fruit-flavored drinks, vegetable drinks, nutritional drinks, energy drinks, sports drinks, soy milk drinks, flavored soy drinks, rice milk-based drinks, flavored milk drinks, yogurt-based drinks, infant formula, tea-based beverages, coffee-based beverages, meal replacement drinks, protein shakes, nutritional supplement beverages, weight management beverages, and combinations thereof.

The edible material comprising the beverage food product can and will vary. Non-limiting examples of suitable edible materials include fruit juices, vegetable juices, skim milk, reduced fat milk, 2% milk, whole milk, cream, evaporated milk, yogurt, buttermilk, chocolate, cocoa powder, coffee, tea, and so forth.

The beverage food product may further comprise natural and artificial sweetening agents (e.g., glucose, sucrose, fructose, maltodextrin, sucralose, aspartame, saccharin, stevia, corn syrup, honey, maple syrup, etc.), flavoring agents (e.g., chocolate, cocoa, chocolate flavor, vanilla extract, vanilla flavor, fruit flavors, etc.), emulsifying or thickening agents (e.g., lecithin, carrageenan, cellulose gum, cellulose gel, starch, gum arabic, xanthan gum, etc.), stabilizing agents, lipid materials (e.g., canola oil, sunflower oil, high oleic sunflower oil, fat powder, etc.), preservatives and antioxidants (e.g., potassium sorbate, sorbic acid, BHA, BHT, TBHQ, rosemary extract, vitamins A, C and E and derivatives thereof, and various plant extracts such as those containing carotenoids, tocopherols or flavonoids having antioxidant properties, etc.), coloring agents, vitamins, minerals, and combinations thereof.

In another embodiment, the food product is a food bar, such as a granola bar, a cereal bar, a nutrition bar, a meal replacement bar, or an energy bar. In still another embodiment, the food product is a cereal-based product. Non-limiting examples of cereal-based food products include breakfast cereals, breakfast bars, pasta, breads, baked products (e.g., cakes, pies, rolls, cookies, crackers), and snack products (e.g., chips, pretzels, etc.). The edible material of a cereal-based food product may be, for example, derived from wheat (e.g., bleached flour, whole wheat flour, wheat germ, wheat bran, etc.), corn (e.g., corn flour, cornmeal, cornstarch, etc.), oats (e.g., puffed oats, oatmeal, oat flour, etc.), rice (e.g., puffed rice, rice flour, rice starch), and so forth. In another embodiment, the food product may be a “solid” dairy-based product. Non-limiting examples of suitable “solid” dairy-based food products include a hard cheese product, soft cheese product, ice cream product, yogurt product, frozen yogurt product, whipped dairy-like product, sherbet, etc. In another embodiment, the food product is a nutritional supplement. The nutritional supplement may be liquid or solid. In another alternate embodiment, the food product is a meat product or a meat analog product. Examples of meat food products include, for example, processed meats, comminuted meats, and whole muscle meat products. The meat material may be animal meat or seafood meat. The meat analog may be a textured vegetable or dairy protein that mimics animal or seafood meat in texture. The meat analog may be part or all of the meat material in a meat food product.

### IV. Definitions

To facilitate understanding of the invention, several terms are defined below.

The term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

The term “degree of hydrolysis” (DH) refers to the percent of specific peptide bonds hydrolyzed (i.e., the number of peptide bonds cleaved out of the total number of peptide bonds present in the intact protein). % DH is estimated using either the trinitrobenzene sulfonic acid (TNBS) or the ortho-phthaldialdehyde (OPA) method. These procedures are accurate, reproducible, and generally applicable procedures for determining DH of a protein hydrolysate.

The term “endopeptidase” refers to an enzyme that hydrolyzes internal peptide bonds in oligopeptide or polypeptide chains. The group of endopeptidases comprises enzyme subclasses EC 3.4.21-25 (International Union of Biochemistry and Molecular Biology enzyme classification system).

The term “exopeptidase” refers to an enzyme that hydrolyzes peptide bonds at or near their amino- or carboxyl termini. The group of exopeptidases comprises enzyme subclasses EC 3.4.11-18 (International Union of Biochemistry and Molecular Biology enzyme classification system).

A “food grade enzyme” is an enzyme that is generally recognized as safe (GRAS) approved and is safe when consumed by an organism, such as a human. Typically, the enzyme and the product from which the enzyme may be derived are produced in accordance with applicable legal and regulatory guidelines.

A “hydrolysate” is a reaction product obtained by bond cleavage. A protein hydrolysate or hydrolyzed protein is a reaction product obtained by peptide bond cleavage of a protein. A protein hydrolysate occurs subsequent to a thermal, chemical, and/or enzymatic reaction. During the reaction, proteins are. broken down into polypeptides, and/or free amino acids. These products may be soluble or insoluble in water or water-based buffer solutions. A hydrolysate should not be confused with a protein composition digested with pepsin-pancreatin.

The “OPA method” as used herein refers to the following procedure: 0.25 g of protein hydrolysate is dissolved in 50 ml of extraction buffer (1% SDS in 0.025 N Sodium Hydroxide, 0.6 mM DTT) by shaking for 5 minutes at 65° C., then cooled to 25° C. The sample is then centrifuged at 5000×g for 5 minutes to remove any undissolved material. Next, 0.2 ml aliquots of the sample, serine standard (3.6 mM in deionized water), and extraction buffer (used as a blank) are transferred (in triplicate) to test tubes, diluted with 10 ml OPA color reagent (0.012M OPA, 0.1M sodium tetraborate, 2% SDS), and vortexed to mix. Reactions are allowed to proceed for 30 minutes, at which time absorbance is measured at 340 nm in a spectrophotometer. Means of each triplicate sample are used to determine % DH as described by Nielsen (Nielsen, P. M et al (2001) “Improved Method for Determining Food Protein Degree of Hydrolysis”, J. Food Sci. 66(5):642-646).

The terms “soy protein isolate” or “isolated soy protein” as used herein refers to a soy material having a protein content of at least about 90% soy protein on a moisture free basis. A soy protein isolate is formed from soybeans by removing the hull and germ of the soybean from the cotyledon, flaking or grinding the cotyledon and removing oil from the flaked or ground cotyledon, separating the soy protein and carbohydrates of the cotyledon from the cotyledon fiber, and subsequently separating the soy protein from the carbohydrates.

The term “soy protein concentrate” as used herein refers to a soy material having a protein content of from about 65% to less than about 90% soy protein on a moisture-free basis. Soy protein concentrate may also contain soy cotyledon fiber, typically from about 3.5% up to about 20% soy cotyledon fiber by weight on a moisture-free basis. A soy protein concentrate is formed from soybeans by removing the hull and germ of the soybean, flaking or grinding the cotyledon and removing oil from the flaked or ground cotyledon, and separating the soy protein and soy cotyledon fiber from the soluble carbohydrates of the cotyledon.

The term “soy flour” as used herein refers to full-fat soy flour, enzyme-active soy flour, defatted soy flour, partially defatted soy flour, and mixtures thereof. Defatted soy flour refers to a comminuted form of defatted soybean material, preferably containing less than about 1% oil, formed of particles having a size such that the particles can pass through a No. 100 mesh (U.S. Standard) screen. The soy cake, chips, flakes, meal, or mixture of the materials are comminuted into soy flour using conventional soy grinding processes. Soy flour has a soy protein content of about 49% to about 65% on a moisture free basis. Preferably the flour is very finely ground, most preferably so that less than about 1% of the flour is retained on a 300 mesh (U.S. Standard) screen. Full-fat soy flour refers to ground whole soybeans containing all of the original oil, usually 18% to 20%. The flour may be enzyme-active or it may be heat-processed or toasted to minimize enzyme activity. Enzyme-activity soy flour refers to full-fat soy flour that has been minimally heat-treated in order not to neutralize its natural enzymes.

The term “soymilk” as used herein, refers to an aqueous mixture of any one or more of the following, finely ground soybeans, soy flour, soy flakes, soy concentrate, isolated soy protein, soy whey protein, and aqueous extracts of any one or more of the following: soybeans, soy flour, or soy flakes wherein insoluble material has been removed. Soymilk may comprise additional components including, for example, fats, carbohydrates, sweeteners, colorants, stabilizers, thickeners, flavorings, acids, and bases.

The term “soymilk powder” as used herein refers to a dewatered soymilk. Soymilk may be dewatered by many processes that include, for example, spray drying, tray drying, tunnel drying, and freeze drying.

The term “simplified trinitrobenzene sulfonic acid (S-TNBS) method” as used herein refers to an accurate, reproducible, and generally applicable procedure for determining DH of a protein hydrolysate. For this method, 0.1 g of the protein hydrolysate is dissolved in 100 mL of 0.025 N NaOH. An aliquot (2.0 mL) of the hydrolysate solution is mixed with 8 mL of 0.05 M sodium borate buffer (pH 9.5). Two mL of the buffered hydrolysate solution is treated with 0.20 mL of 10% trinitrobenzene sulfonic acid, followed by incubation in the dark for 15 minutes at room temperature. The reaction is quenched by adding 4 mL of a 0.1 M sodium sulfite-0.1 M sodium phosphate solution (1:99 ratio), and the absorbance read at 420 nm. A 0.1 mM glycine solution is used as the standard. The following calculation is used to determine the percent recovery for the glycine standard solution: [(absorbance of glycine at 420 nm—absorbance of blank at 420 nm)×(100/0.710)]. Values of 94% or higher were considered acceptable. (Jens Adler-Nissen (1979) “Determination of the Degree of Hydrolysis of Food Protein Hydrolysates by Trinitrobenzenesulfonic Acid,” J. Agric. Food Chem., 27(6):1256-1262).

When introducing elements of the present invention or an embodiment(s) thereof, the articles “a,” “an,” “the,” and “said” are intended to mean that there are one or more of the elements. The terms “comprising” and all its forms and tenses (including, for example, comprise and comprised) is synonymous with “including,” “containing,” or “characterized by,” and is inclusive or open-ended language and does not exclude any additional, unrecited element, step, or ingredient. The terms “consisting” and all its forms and tenses (including, for example, consist and consisted) is closed language and excludes any element, step, or ingredient not specified. The terms “consisting essentially of” and all its forms and tenses limits the scope of the invention to the specified element, step, or ingredient and those that do not materially affect the basic and novel characteristic(s) of the claimed invention. Applicants note that certain embodiments recite the transitional phrase comprising; wherever Applicants have recited this transitional phrase, the transitional phrase consisting of or consisting essentially of have also been contemplated by Applicants and form part of the invention.

As various changes could be made in the above compositions, products and methods without departing from the scope of the invention, it is intended that all matter contained in the above description and in the examples given below, shall be interpreted as illustrative and not in a limiting sense.

### V. EXAMPLES

### Example 1: TL1 Protein Hydrolysate

TL1 hydrolysates were prepared as follows. 3L of aqueous Supro® 760 isolated soy protein (ISP) protein solution at concentration of 8% solids was prepared by resuspending 240 g of protein (lot#M310007644) into 2760 g of warm tap water using moderate propeller blade mixing at room temperature. The suspension was heated to 50° C. on a hot plate, adjusted to pH 8 with 1N food-grade NaOH, and split into 4 equal 650 ml aliquots. TL1 enzyme (Novozymes NS12001) was then added to each at a final concentration of 150 mg enzyme protein/Kg solids, and the suspensions incubated at 50° C. using a Hanson Research Dissolution Station. Duplicate samples were transferred to 1L beakers at 30 and 60 minutes intervals, covered with aluminum foil, and heated to 80° C. on a hot plate. Once at temperature, the foil was removed and the heating continued for 5 minutes to inactivate the TL-1 enzyme. Finally, the hydrolysate was cooled to 40° C. using a wet ice bath, then frozen at −40° C. prior to lyophilization.

### Example 2: Neutrase Protein Hydrolysate

Neutrase hydrolysates were prepared as follows. 1380 g of tap water was placed in a 2L beaker, then heated to 50° C. in a water bath. 120 g of Supro® 760 (lot#M310007644) ISP was then resuspended in the water with moderate stirring, and the pH of the suspension adjusted to 7 or 8.5 with 1N food-grade NaOH. Next, Neutrase® 1.5 MG (lot#PW200921) was added to the suspension to a final concentration of 75 or 150 mg enzyme protein/Kg solids, and the suspension incubated at 50° C. for 120 minutes. For some hydrolysates, pH was held constant during the hydrolysis period using a Mettler Toledo DL50 Graphix Titrator to meter in NaOH. At the end of the incubation period, the reaction was stopped by moving the beaker to a hot plate and heating the hydrolysate to 80° C. for 5 minutes. Sample was then chilled in a water and ice bath, then frozen at −40° C. and lyophilized to dryness.

### Example 3: SP-1 Protein Hydrolysate

SP-1 hydrolysates were prepared as follows. 920 g of tap water was placed in a 2L beaker, then heated to 70° C. in a water bath. 80 g of Supro® 760 (lot#P220014935) ISP was then resuspended into the water with moderate stirring, and the pH of the suspension adjusted to 9 with 1N food-grade NaOH. Next, the serine protease SP-1 was added to the suspension to a final concentration of 100 mg enzyme protein/Kg solids, and the suspension incubated at 70° C. for 30 minutes. At the end of the incubation period, the reaction was stopped by moving the beaker to a hot plate and heating the hydrolysate to 80° C. for 5 minutes. Sample was then chilled in a water and ice bath, then frozen at −40° C. and lyophilized to dryness.

### Example 4: S2 Protein Hydrolysate

S2 hydrolysates were prepared as follows. 1150 g of tap water was placed in a 2L beaker, then heated to 70° C. in a water bath. 120 g of Supro® 760 (lot#M310007644) ISP was then resuspended into the water with moderate stirring, and the pH of the suspension adjusted to 7 with 1N food-grade NaOH. Next, a protease of the subtilisin family, S2, was added to the suspension to a final concentration of 75 or 150 mg enzyme protein/Kg solids, and the suspension incubated at 70° C. for either 30 or 120 minutes. pH was held constant during the hydrolysis period using a Mettler Toledo DL50 Graphix Titrator to meter in NaOH. At the end of the incubation period, the reaction was stopped by moving the beaker to a hot plate and heating the hydrolysate to 80° C. for 5 minutes. Sample was then chilled in a water and ice bath, then frozen at −40° C. and lyophilized to dryness.

### Example 5: S1 Protein Hydrolysate

S1 hydrolysates were prepared as follows. 1150 g of tap water was placed in a 2L beaker, then heated to 70° C. in a water bath. 100 g of Supro® 760 (lot#M310007644) ISP was then resuspended into the water with moderate stirring, and the pH of the suspension adjusted to either 8.0 or 9.0 with 1N food-grade NaOH. Next, a protease of the subtilisin family, S1, was added to the suspension to a final concentration of 100 mg enzyme protein/Kg solids, and the suspension incubated at 70° C. for 120 minutes. pH was held constant during the hydrolysis period using a Mettler Toledo DL50 Graphix Titrator to meter in NaOH. At the end of the incubation period, the reaction was stopped by heating the hydrolysate to 90° C. using a steam kettle, then holding at temperature for an additional 2 minutes. Sample was then chilled in a water and ice bath, then frozen at −40° C. and lyophilized to dryness.

The following table gives the hydrolysis conditions and the degree of hydrolysis (as determined using the OPA method of Nielsen, Petersen, and Damdmann (2001), J. Food Sci 66(5):642-646) for various protein hydrolysate compositions of this invention.

| | | | | | | | pH Stat | |
|---|---|---|---|---|---|---|---|---|
| Sample # | Enzyme | Dose | Units | Time (Min) | Temperature (C.) | Hydrolysis_pH | (Y/N) | % DH |
| 1 | Neutrase | 75 | mg/kg | 120 | 50 | 7.00 | Y | 4.09 |
| 2 | Neutrase | 150 | mg/kg | 120 | 50 | 7.00 | Y | 7.27 |
| 3 | Neutrase | 75 | mg/kg | 30 | 50 | 8.50 | N | 4.52 |
| 4 | TL-1 | 150 | mg/Kg | 60 | 50 | 8.00 | N | 2.78 |
| 5 | TL-1 | 150 | mg/Kg | 30 | 50 | 8.00 | N | 3.24 |
| 6 | S1 | 100 | mg/Kg | 120 | 70 | 8.00 | Y | 1.58 |
| 7 | S1 | 100 | mg/Kg | 120 | 70 | 9.00 | Y | 0.15 |
| 8 | SP-1 | 100 | mg/Kg | 30 | 70 | 9.00 | N | 4.99 |
| 9 | S2 | 75 | mg/Kg | 30 | 70 | 9.00 | Y | 2.18 |
| 10 | S2 | 150 | mg/Kg | 120 | 70 | 9.00 | Y | 2.28 |

### Example 6: Size Exclusion Chromatography to Determine MW Distribution of Protein Hydrolysates

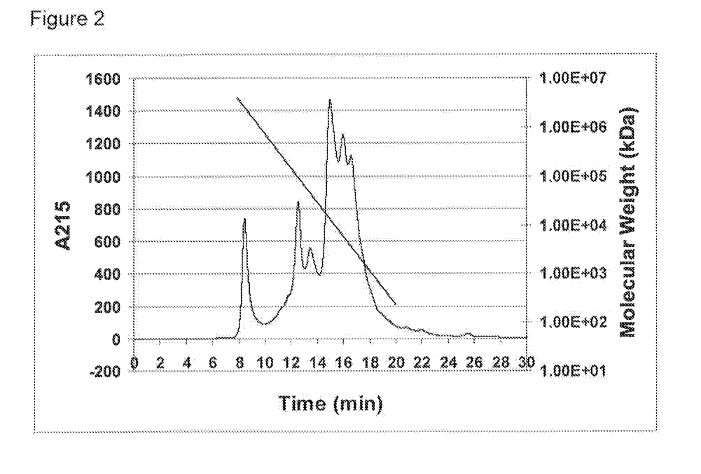

A representative sample of each of the hydrolysates prepared as described in the Examples was resuspended in phosphate-buffered saline (PBS, pH 7.4, Sigma cat#P5368) at a solids concentration of 2.5%. Insoluble material was removed by centrifugation at 16,000×g for 10 minutes, and the resulting supernatant passed through a 0.45 micron syringe filter to remove any remaining fines. 30 μl of each sample was then sequentially injected onto a Shodex Protein KW-803 Size Exclusion Chromatography column (Shodex, Inc.) pre-equilibrated in PBS, and separated at a flow rate of 0.7 ml/min. Peptides were detected at 215 nm, which was continuously recorded, and plotted against time. Retention times for a set of molecular weight standards were determined in a similar fashion. These were plotted vs. the log molecular weight of each standard to generate a standard curve. Using this curve, retention time ranges for select molecular weight ranges was determined. Molecular weight distribution data for the peptides present in each sample was then determined by calculating the total area under the curve for a specified molecular weight range, dividing by the total area under the curve, and multiplying by 100. FIG. 2 depicts a representative chromatogram from this experiment. Data for certain protein hydrolysate compositions of the invention are provided in the following table.

| | | | | MW Range | | | | | |
|---|---|---|---|---|---|---|---|---|---|
| Sample # | Enzyme | DoSe | Units | >20 kDa | 10-20 kDa | 5-10 kDa | 2-5 kDa | 1-2 kDa | <1 kDa |
| 1 | Neutrase | 75 | mg/kg | 38% | 15% | 17% | 19% | 7% | 5% |
| 2 | Neutrase | 150 | mg/kg | 33% | 13% | 18% | 22% | 9% | 6% |
| 3 | Neutrase | 75 | mg/kg | 36% | 17% | 17% | 19% | 6% | 5% |
| 4 | TL-1 | 150 | mg/Kg | 39% | 16% | 17% | 17% | 6% | 4% |
| 5 | TL-1 | 150 | mg/Kg | 44% | 15% | 17% | 15% | 6% | 3% |
| 6 | S1 | 100 | mg/Kg | 35% | 21% | 20% | 15% | 5% | 3% |
| 7 | S1 | 100 | mg/Kg | 54% | 20% | 12% | 9% | 3% | 2% |
| 8 | SP-1 | 100 | mg/Kg | 28% | 15% | 21% | 24% | 8% | 5% |
| 9 | S2 | 75 | mg/Kg | 33% | 18% | 20% | 19% | 7% | 4% |
| 10 | S2 | 150 | mg/Kg | 31% | 16% | 20% | 21% | 8% | 4% |

### Example 7: Solubility of Protein Hydrolysates

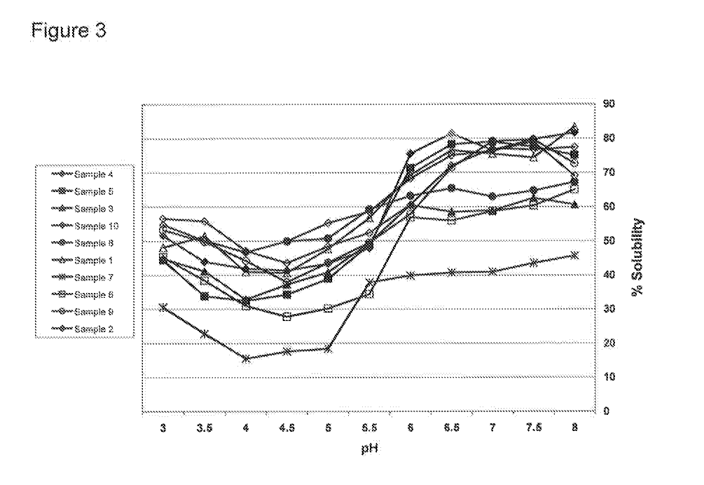

The solubility of each hydrolysate was determined at several pHs between 3 and 8.5. 1.25 g of each hydrolysate was combined with 48.75 g of deionized water and mixed on a stir plate until the material was completely resuspended. The pH of each sample was then adjusted to between 3.0 and 8.5 using either 1N HCl or 1N NaOH. 25 ml of the resultant slurries were next centrifuged at 500×g for 10 minutes. 5 ml of each suspension and 5 ml of each supernatant were then transferred to pre-weighed aluminum pans and incubated overnight at 130° C. to dry. Weights of each sample were recorded after correcting for the pan weight. Solubility was determined by dividing the weight of the solids in the soluble portion of each sample by the weight of the total solids. Solubility curves for each sample were prepared by plotting % solubility vs. pH. Data is depicted in FIG. 3.

### Example 8: CCK and GLP-1 Assay

To assay for CCK or GLP-1 releasing activity, the protein hydrolysate or protein powders (or lyophilized pepsin-pancreatin digested hydrolysates or proteins) were hydrated overnight in Dulbecco's phosphate buffered saline (D-PBS). The resultant protein solutions were centrifuged at 16,000×g for 30 minutes at 4° C. to remove any insoluble protein. Supernatants were assayed for total protein content by the Bicinchoninic Acid method (Pierce® BCA) according to the manufacturer's instructions. The culture supernants were added to the media of STC-1 cells (passage 25 to 28) in 1:1 D-PBS: Dulbecco's Modified Eagle's Medium (DMEM-high glucose) at a protein concentration of 2 mg/mL or as indicated in the various examples. Hydrolysates were incubated at 37° C. with the STC-1 cells for 4 hours (CCK release) or 2 hours (GLP-1 release). When pepsin and pancreatin digested proteins or hydrolysates were added to cells, enzyme controls were added at equivalent dilutions of the control reaction mixture (which included pepsin and pancreatin in the absence of protein substrate). Fatty acid-free bovine serum albumin (BSA) was added at 2 mg/mL as a negative control. Positive controls for the CCK releasing assay were a soy protein hydrolysate (2 mg/mL) previously shown to have significant CCK releasing activity (the hydrolyzed soy protein composition FXP950; see, for example, PCT Application No. PCT/US2009/069867) and 100 nM phorbol-12-myrisate 13-acetate (PMA) (secondary control). For the GLP-1 releasing assay, BSA and FXP950 (both 2 mg/mL) were used as the negative and positive control, respectively. In addition 10 μM forskolin and 100 nM PMA were used as secondary assay controls for GLP-1 measurements. After the hydrolysates and controls were incubated with the STC-1 cells, the culture media were harvested. For the CCK assay 0.6 trypsin inhibitory units (TIU)/mL aprotinin was added and for the GLP-1 assay 0.55 TIU/mL aprotinin and 290 μM dipeptidyl peptidase-4 inhibitor (DPP-IVi) were added to the media to prevent proteolyic degradation of the peptide hormones. The media were then centrifuged at 500×g for 5 minutes at 4° C. to pellet any cell debris and the supernatants were transferred to 96 well V-bottom microtiter plates and stored at −80° C. until assayed for CCK or GLP-1 by ELISA. Concentrations of CCK and GLP-1 released by the STC-1 cells into the media were assayed using commercially available immunoassay kits from Phoenix Pharmaceuticals, Burlingame, Calif. (catalogue numbers EK-069-04 and EK-028-11, respectively). Assays were performed according to the manufacturer's instructions using a more extensive standard curve covering a range of concentrations from 0.4 to 1000 pg/well. Absorbance is measured at a wavelength of 450 nm.

Results are expressed as the % CCK released into the media of STC-1 cells stimulated by the test samples (undigested or pepsin-pancreatin digested hydrolysates or intact proteins) compared to CCK released by the positive control FXP 950 soy protein hydrolysate (which was set at 100%). % CCK released into the media in each cell culture well is calculated as follows:

$$\% CCK release = \frac{( ng {CCK}_{test hydrolysate / protein} - ng {CCK}_{BSA} )}{( ng {CCK}_{FXP 950 control} - ng {CCK}_{BSA} )} \times 100$$

where ng CCK_{BSA} is the ng/well CCK released in response to BSA alone and ng CCK_{FXP950 control} is the ng/well CCK released in response to the control hydrolysate, FXP950. Similarly, % GLP-1 released into the media in each cell culture well is calculated as follows:

$$\% GLP \text{-} 1 release = \frac{( ng GLP \text{-} {1}_{test hydrolysate / protein} - ng GLP \text{-} {1}_{BSA} )}{( ng GLP \text{-} {1}_{FXP 950 control} - ng GLP \text{-} {1}_{BSA} )} \times 100$$

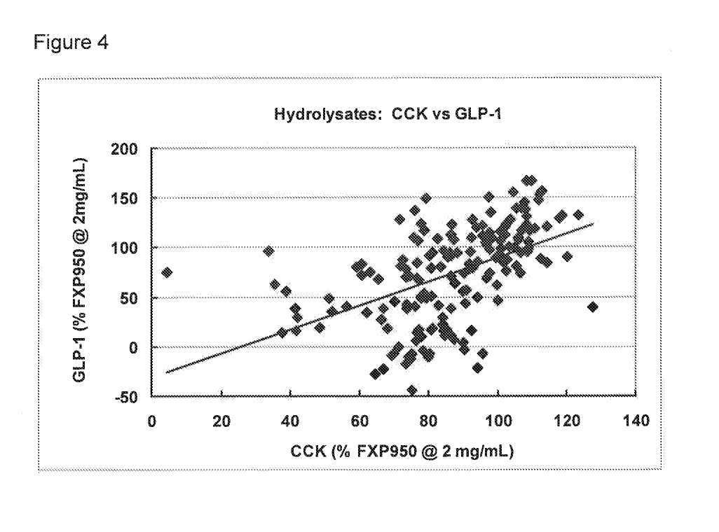

The mechanisms whereby peptides stimulate CCK and GLP-1 have not been clearly identified, but it would appear that the mechanisms that stimulate each would be different given that two different enteroendocrine cells give rise to these two different peptide hormones. Considering this, it was unexpected that peptides from a single hydrolysate would show a significant ability to induce the release of both CCK and GLP-1. Nonetheless, we show that a soy protein hydrolysate (at a concentration of 2 mg/mL), previously shown to induce CCK release, also induces GLP-1 release to a level comparable to that of 10 μM forskolin which has been shown to stimulate GLP-1 in STC-1 cells (Islam D et al. (2009) Am J Physiol Endocrinol Metab 296: E174-E181). Moreover, we show that a number of soy protein hydrolysates generated through specific proteolytic enzyme cleavage and conditions do exhibit an enhanced ability to induce the release of both CCK and GLP-1 from STC-1 cells compared to other hydrolysates. A wide range of CCK and GLP-1 inducing abilities are shown with 170 different soy protein hydrolysates in FIG. 4. Soy protein hydrolysates were prepared as described herein and 2 mg/mL of the soluble protein fractions were applied to STC-1 cells and incubated for 2 (GLP-1) or 4 hours (CCK). Culture media was assayed for GLP-1 or CCK and the results are expressed as a percent GLP-1 or CCK released compared to that released by 2 mg/mL FXP950 (control soy protein hydrolysate). There is only a very limited correlation (r²=0.2453) of CCK and GLP-1 inducing activity suggesting that the peptide signals that induce both hormones only overlap in a unique subset of peptide hydrolysates, which supports unexpected results whereby a hydrolysate induces the release of both CCK and GLP-1.

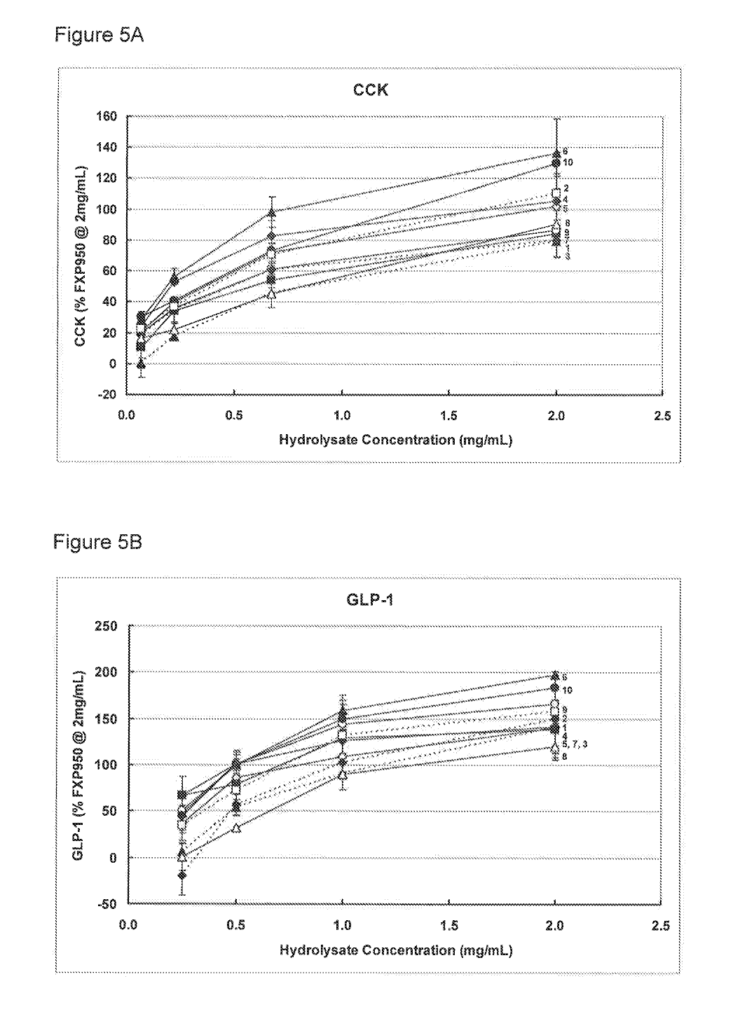

FIG. 5 demonstrates that selected hydrolysates that showed enhanced release of both CCK and GLP-1 do so in a dose responsive manner. Without being bound by theory, it is believed that the resultant dose response curves suggest a saturable, possibly receptor mediated, mechanism of induction of both CCK and GLP-1 by the hydrolysates. Again, without being bound by theory, it is believed that due to absolute differences in the abilities of the various hydrolysates to induce CCK and GLP-1 that each hydrolysate is composed of differing amounts of specific peptides required for induction or that the overall potency of the peptides to induce the hormones varies for each hydrolysate. Therefore, it can be concluded that differences in the proteolytic specificities used to generate the hydrolysates accounts for the relative differences in bioactivity of the latter and that specific peptide sequences are likely responsible for maximal CCK and GLP-1 induction.

### Example 9: Wheat Bread

The following example relates to a wheat bread product (˜265 Kcal/100 g) that comprises a protein hydrolysate of the invention.

Wheat bread is formed according to typical industry processing techniques using the “Sponge and Dough” method following the step-by-step process below. The following table is an example of ingredients and the amounts used in grams.

| | Control | Hydrolysate |
|---|---|---|
| Ingredients | (g) | (g) |
| Sponge | | |
| Whole Wheat Flour, Ultra fine | 700 | 650 |
| Soy Protein Hydrolysate | 0 | 77.6 |
| Instant Dry Yeast | 12 | 12 |
| Vital Wheat Gluten | 50 | 50 |
| Mineral Yeast Food (Non-Brominated Type) | 5 | 5 |
| Grindsted SSLP55 Veg, Danisco | 5 | 5 |
| Water <4° C. | 505 | 477.4 |
| Sponge Total | 1277 | 1277 |
| Dough | | |
| Whole White Flour, Ultra Fine | 265.8 | 240 |
| Soy Protein Hydrolysate | 0 | 38.8 |
| Salt | 20 | 20 |
| Honey 6%, = 4.944% sugar | 60 | 60 |
| Brown Sugar | 20 | 20 |
| Yeast, compressed | 10 | 10 |
| HFCS-42 4%, = 2.84% sugar | 40 | 40 |
| Calcium propionate | 2.5 | 2.5 |
| Monoglyceride, GMS90 | 5 | 5 |
| Bake Soft C 1650, Caravan Ingredients | 0.31 | 0.31 |
| Ascorbic Acid 60 ppm, | 11 | 11 |
| Lecithin, SOLEC ™ 3FUB | 10 | 10 |
| Soybean Oil | 34.2 | 34.2 |
| Water | 165.7 | 152.7 |
| Total Dough Weight | 644.51 | 644.51 |
| Total of dough and sponge | 1791.01 | 1791.01 |

The ingredients are combined and processed according to the following steps to produce a wheat bread product:

### I. Production of Sponge:

A. The Sponge ingredients were combined and mixed for 1 minute on medium and 3 minutes on speed 2 using a Hobart A-200 mixer with McDuffie attachment.
B. During the combining of the Sponge ingredients the temperature was maintained at 26° C.
C. The sponge is then allowed to ferment for 2.5 to 3 hours at 35° C. and 85% relative humidity (RH).

### II. Production of Dough:

A. The dough ingredients are combined in a mixing bowl, and mixed at speed 1 for 1 minute; next the sponge mixture is added and mixed for 4 minutes on speed 2.
B. The dough mixture is allowed to rest for 10 minutes.
C. The dough mixture is separated into 570 g round pieces.
D. The dough mixture pieces are placed on a sheet and molded.
E. The dough mixture pieces are proofed for 60 minutes at 43° C. and 90% RH.
F. Finally the dough mixture pieces are baked in a preheated oven at 221° C. for 22 minutes.

The results are a wheat bread composition that has an increased quantity of soy protein hydrolysate at about 10% total energy, on a ready-to-consume basis, but that retains the taste, structure, aroma, and mouthfeel of typical wheat bread products currently on the market.

### Example 10: Cracker

The following example relates to a cracker (˜375 Kcal/100 g) that comprises a protein hydrolysate of the invention.

The crackers are formed according to the following process. The following table is a list of ingredients by weight in grams.

| | Control | Soy Protein |
|---|---|---|
| Ingredients | (kg) | Hydrolysate |
| Flour, pastry (soft wheat) | 21.9500 | 18.6900 |
| Soybean Oil | 4.3400 | 4.3400 |
| Soy Protein Hydrolysate | 0.0000 | 3.2600 |
| Granulated sugar | 1.7500 | 1.7500 |
| High fructose corn syrup (55%) | 1.3200 | 1.3200 |
| Skimmed milk powder | 0.1100 | 0.1100 |
| Salt | 0.2200 | 0.2200 |
| Sodium Bicarbonate | 0.1900 | 0.1900 |
| Monocalcium Phosphate | 0.2000 | 0.2000 |
| Ammonium bicarbonate | 0.3300 | 0.3300 |
| Enzyme (crackerase) | 0.0036 | 0.0036 |
| Butter flavor | 0.1800 | 0.1800 |
| Water (32° C.) | 5.6900 | 5.0000 |
| Total | 30.59 | 30.59 |

The ingredients are combined and processed according to the following steps to produce crackers:

### I. Production of Crackers

A. All dry ingredients are combined and blended for 5 minutes.
B. The remaining ingredients are added to the dry ingredient mixture, with the exception of ammonium bicarbonate and enzyme, which are pre-dissolved in the formulation water and held back.
C. The ammonium bicarbonate and enzyme solution is added at this point, and then the blend is mixed for an extended period of time, 10 to 15 minutes in order for the dough to form.
D. The dough is allowed to set and relax for 30 minutes at room temperature.
E. After the dough sets, it is divided into 75 g pieces and rounded slightly by conventional means.
F. The round dough pieces are next pressed by conventional means into discs approximately 12 mm (0.5 inch) thick.
G. The dough discs are processed through a sheeting machine with a gap 1 setting at 4.5. The dough pieces are then folded into thirds with the edges trimmed.
H. The dough pieces are next rotated 90 degrees and passed through the sheeting machine gap 1 again with the gap set at 2.5. The dough pieces are then folded into thirds with the edges trimmed
I. The dough pieces are next rotated 90 degrees, slightly dusted with flour, and passed through the sheeting machine gap 2 with the gap setting at 1.5 to 1.75.
J. The dough pieces are cut into the desired shape and each piece is pierced by conventional means so the crackers are crispy when finished.
K. The dough pieces are baked at 230° C. (450° F.) for 6 minutes, removed from the oven, cooled and placed in a sealed plastic bag.

The results are crackers that have an increased an increased quantity of soy protein hydrolysate at about 10% of the total energy value of the crackers, but retaining the taste, structure, aroma, and mouthfeel of typical cracker products currently on the market.

### Example 11: Baked Bar

| | Ingredient | % | g |
|---|---|---|---|
| Syrup | Corn Syrup, 63 D.E | 19.46% | 58.38 |
| | High Fructose Corn Syrup (55%) | 14.00% | 42 |
| | Glycerine | 15.00% | 45 |
| | Chocolate Flavour | 0.41% | 1.23 |
| | Vanilla Flavour | 0.10% | 0.3 |
| Powder | Supro ® Isolated Soy Protein (430) | 14.95% | 44.85 |
| Blend | Soy Protein Hydrolysate | 14.95% | 44.85 |
| | Rice Syrup Solids (Corn Syrup Solids) | 16.72% | 50.16 |
| | Cocoa powder 12% fat | 3.81% | 11.43 |
| | Vitamin and Mineral premix | 0.52% | 1.56 |
| | Salt | 0.08% | 0.24 |
| | Batch total | 100.00% | 300 |

The following example relates to a baked bar (˜405 Kcal/100 g) that comprises a protein hydrolysate of the invention.

The baked bar is formed according to the following process. The following table is a list of ingredients and the amount used in kilograms.

The ingredients are combined and processed according to the following steps to produce the baked bar:

The isolated soy protein, hydrolyzed soy protein, rice syrup solids (available from Natural Products, Lathrop, Calif.), cocoa powder (available from DeZaan, Milwaukee, Wis.), vitamin & mineral premix (available from Fortitech®, Schenectady, N.Y.), and 1.6 grams of salt are added to a Winkworth mixer (available from Winkworth Machinery, Ltd., Reading, England) and mixed at a speed of 48 revolutions per minute (rpm) for one minute. In a separate container, a second mixture containing the liquid sugar syrups, the glycerine and the liquid flavoring agents is heated to a temperature of 37.8° C. (100° F.) by microwaving on high power for about 45 seconds. The liquid sugar syrup consists of a 55:45 blend of 63 DE corn syrup (available from Roquette®, LESTREM Cedex, France) and high fructose corn syrup 55 (available from International Molasses Corp., Rochelle Park, N.J.) and 566.0 grams glycerine. The liquid flavouring agents consist of 4.1 grams Edlong® Chocolate flavour 610 (available from The Edlong® Corporation, Elk Grove Village, Ill.), 4.1 grams Edlong® Chocolate flavour 614 (available from The Edlong® Corporation, Elk Grove Village, Ill.), and vanilla flavouring (available from Sethness Greenleaf, Inc., Chicago, Ill.). The heated second mixture is then mixed into the first mixture in a Winkworth mixer at a speed of 48 rpm for three minutes and forty-five seconds. The resulting dough is then sheeted out onto a marble slab and bars are cut into pieces weighing from about 45 grams to about 55 grams (the bar pieces are about 102 millimetres in length, about 10 millimetres in height, and about 35 millimetres wide).

The result is a baked bar enriched with a soy protein hydrolysate wherein the soy protein hydrolysate contributes about 15% of the total energy, measured in kilocalories, of the baked bar.

### Example 12: Formulated Soymilk

The following example relates to a soymilk (˜80 Kcal/240 g serving) that comprises a protein hydrolysate of the invention.

The soymilk is made according to the following process. The following table is a list of ingredients and the amount used in grams.

| | Formula 1 | | Formula 2 | |
|---|---|---|---|---|
| | (%) | g/ | (%) | g/ |
| Ingredients | as is | 10000 g | as is | 10000 g |
| Deionised Water | 88.529 | 885.29 | 88.469 | 884.69 |
| Supro ® 120 | 3.422 | 34.22 | 0.112 | 1.12 |
| Soy Protein Hydrolysate | 0 | 0 | 3.31 | 33.1 |
| Sugar | 2.75 | 27.5 | 2.75 | 27.5 |
| Maltodextrin, 15DE | 3.521 | 35.21 | 3.521 | 35.21 |
| Potassium Citrate | 0.2 | 2 | 0.2 | 2 |
| Magnesium Phosphate, | 0.038 | 0.38 | 0.038 | 0.38 |
| dibasic | | | | |
| Salt | 0.03 | 0.3 | 0.03 | 0.3 |
| Iota-carrageenan | 0.01 | 0.1 | 0 | 0 |
| Lambda carrageenan | 0 | 0 | 0.07 | 0.7 |
| Cellulose gum | 0.25 | 2.5 | 0.25 | 2.5 |
| Sunflower oil High Oleic | 1 | 10 | 1 | 10 |
| Vanilla flavour | 0.25 | 2.5 | 0.25 | 2.5 |
| Total: | 100 | 1000 | 100 | 1000 |

The ingredients are combined and processed according to the following steps to produce the soymilk:

### I. Production of Soymilk

A. The soy protein ingredient (Supro® 120 and/or soy protein hydrolysate) is dissolved in water at 38° C. (100° F.) using moderate shear. Food grade antifoam is added when foam levels became problematic, and mixing continues for another 30 minutes.
B. The temperature is raised to 77° C. (170° F.). Mixing continues at low speed for 15 minutes to form the protein slurry.
C. The protein slurry is then homogenized at 200 bar (2800 psi).
D. The appropriate amount of protein slurry per batch is weighed out.
E. Sucrose, maltodextrin, stabilizer, salt and magnesium phosphate are dry blended together and then dispersed into the protein slurry. Mixing continues and the temperature is maintained at 74° C.-77° C. (165° F.-170° F.) for 10 minutes.
F. Sunflower oil is added to the slurry mixing continued at moderate speed until a homogeneous appearance develops (approximately 3 minutes). The pH is adjusted to fall within the range 7.0-7.2 using either 50% citric acid or 45% KOH, whichever is necessary and then the product is heat processed.
G. Heat processing conditions for the Ultra High Temperature (UHT) process are as follows:
   i. The product is homogenized at 500 psi (35 Bar) second stage; 2500 psi (173 Bar) first stage and then preheated to 104° C. (220° F.) and then heated indirectly to 141° C. (286° F.) for 6 seconds.
   ii. Product is cooled firstly to 72° C. (162° F.) and then to 3° C. (37° F.) and immediately packaged aseptically in a laminar air flow cabinet in 250 ml sterilized bottles.
   iii. Bottles are packed in ice-water and then passed into refrigerated storage.

The result is a neutral pH, formulated soymilk that delivers a soy protein hydrolysate at about 35% of energy.

### Example 13: Combination Dairy/Soy Beverage

The following example relates to a combination dairy/soy beverage that comprises a protein hydrolysate of the invention.

Formula:

| | Dairy/Soy | Dairy/Soy |
|---|---|---|
| | Beverage | Beverage |
| INGREDIENTS | (%) | (g/1000 g) |
| Water, Tap | 44.11 | 441.10 |
| Soy protein hydrolysate | 1.28 | 12.80 |
| Supro ® Plus 651 | 0.54 | 5.40 |
| Sugar | 1.75 | 17.50 |
| Maltodextrin, 15DE | 1.76 | 17.60 |
| Mixed Carrageenans | 0.01 | 0.10 |
| Cellulose Gum | 0.01 | 0.10 |
| Vanilla flavor | 0.04 | 0.40 |
| Sunflower oil | 0.50 | 5.00 |
| 1% fat milk | 50.00 | 500.00 |
| Total | 100.00 | 1000.00 |

Process:
A. Tap water at 20-25° C. is added to a mixing vessel of suitable size for the batch. The soy proteins are dispersed in the water with medium shear. Food grade antifoam is added, if necessary to control foaming.
B. The slurry is heated to 70-80° C. and then homogenized at 200 bar (2800 psi).
C. The carrageenans and the cellulose gum are dry blended with a portion of the sugar and added to the protein slurry. The slurry is then heated to maintain a temperature of 80° C.
D. The rest of the sugar and the maltodextrin are then added to the process tank and mixed thoroughly until dispersed and dissolved.
E. The oil and the flavor are added to the batch and the mix is vigorously agitated to form a pre-emulsion.
F. The batch is homogenized using a piston-type homogenizer in two stages at 180 bar (2500 psi) and 30 bar (500 psi).
G. The homogenized batch is cooled to ±5° C. before the 1% fat milk is weighed and added. Gentle mixing suffices to render the blend homogeneous.
H. The dairy/soy beverage is U.H.T (ultra high temperature) processed by indirect heating to 145° C. for 6 seconds, then cooled to <5° C. and packaged into sterile, 500 ml containers under laminar, filtered air flow.

The result is a combination soy/dairy beverage in which about 20% of calories are derived from a soy protein hydrolysate.

### Example 14: Neutral Dry Blended Beverage

The following example relates to a neutral dry blend beverage that comprises a protein hydrolysate of the invention.

Formula:

| | | Batch size | |
|---|---|---|---|
| | g/serving | 500 g | |
| Ingredients: | 50 | % | g/batch |
| Supro ® XT 219D | 3.94 | 7.88 | 39.40 |
| Soy protein hydrolysate | 15.51 | 31.02 | 155.10 |
| Whey Protein Isolate (WPI) | 9.00 | 18.00 | 90.00 |
| Sucrose | 9.00 | 18.00 | 90.00 |
| Digestion resistant maltodextrin | 6.00 | 12.00 | 60.00 |
| Fat Powder (65.5% Fat) | 2.88 | 5.75 | 28.75 |
| Xanthan gum | 0.29 | 0.57 | 2.85 |
| Stevia | 1.37 | 2.73 | 13.65 |
| Potassium Citrate | 0.58 | 1.15 | 5.75 |
| Vitamin Premix | 0.23 | 0.46 | 2.30 |
| Vanilla Flavour | 1.22 | 2.44 | 12.20 |
| Total: | 50.00 | 100.00 | 500.00 |

Process:
A. The protein ingredients Supro® XT 219D, soy protein hydrolysate, and WPI are added to a v-blender and blended for 10 minutes.
B. The rest of the ingredients are added to the blender and the mix is blended for a additional 10 minutes.
C. The powder mix is discharged from the blender and packaged into individual sachets that are then heat sealed. Approximately 50 g of mixture is placed in each sachet.
D. The resulting product is stirred or shaken into 230 ml (8 fluid ounces) of water until smooth (several minutes) to replace a meal as part of a weight loss program. The product delivers a soy protein hydrolysate at about 35% of energy.

### Example 15: High Protein Dry Blended Beverage

Formula:

| Ingredient | % | g |
|---|---|---|
| Soy Protein Hydrolysate | 36.00 | 720.00 |
| Supro ® 219D | 50.50 | 1010.00 |
| Maltodextrin M150 | 8.32 | 166.40 |
| Guar HV (Guar, High Viscosity) | 0.05 | 1.00 |
| Vitamin and mineral premix | 0.09 | 1.80 |
| Defatted Cocoa Powder | 4.68 | 93.60 |
| Chocolate Flavor | 0.32 | 6.40 |
| Sucralose | 0.01 | 0.20 |
| Acesulfam-K | 0.03 | 0.60 |
| TOTAL | 100.0 | 2000.00 |

Process:
A. All of the ingredients are added to the blender and the mix is blended for 10 minutes
B. The powder mix is discharged from the blender and packaged into 1 Kg multi-layer cans and sealed.
C. The resulting product is stirred or shaken at a rate of about 50 g into 230 ml (8 fluid ounces) of water until smooth (several minutes) to serve as a protein supplement for athletes in training. It delivers a soy protein hydrolysate at an amount of about 35% of energy of the product.

### Example 16: Meal Replacement Beverage

The following example relates to a meal replacement beverage that comprises a protein hydrolysate of the invention.

Formula:

| | | weight per |
|---|---|---|
| | | 10000 g |
| Ingredient | % | batch (g) |
| Deionised Water | 79.58 | 7948 |
| Sodium Caseinate | 1.05 | 135 |
| Calcium Caseinate | 1.05 | 135 |
| Soy Protein Hydrolysate | 3.2 | 270 |
| Sucrose | 7 | 700 |
| Corn syrup solids (25DE) | 4.5 | 450 |
| Soybean oil | 0.8 | 80 |
| Canola oil | 0.75 | 75 |
| Corn oil | 0.7 | 70 |
| Lecithin, deoiled | 0.12 | 12 |
| Tricalcium phosphate | 0.2 | 20 |
| Magnesium phosphate, dibasic | 0.21 | 21 |
| Sodium chloride | 0.1 | 10 |
| Mixed Carrageenans | 0.01 | 1 |
| Cellulose Gum | 0.5 | 50 |
| Vitamin Premix | 0.07 | 7 |
| Vanilla Flavour | 0.16 | 16 |
| Total | 100 | 10000 |

Process:
A. Process water (20-25° C.) is added to a process tank. The soy protein hydrolysate is dispersed into the water using medium shear to form a protein slurry. Food grade antifoam is used as necessary to break up foam.
B. The protein slurry is heated to 70-80° C. and homogenized at 200 bar (2800 psi). The pH is adjusted to pH 7.0-7.2.
C. The carrageenan and the cellulose gum are dry blended with a portion of the sugar and added to the protein slurry.
D. The caseinates are dry blended with the rest of the sugar and added to the process tank. The caseinates are allowed to hydrate (10 minutes).
E. The remaining carbohydrates and minerals are added to the process tank and mixed for 5 minutes.
F. The oil and lecithin are mixed separately, heated to 60° C. (140° F.), then added to the process tank and mixed for 5 minutes.
G. The vitamin/mineral premix and flavor are added and mixed for 2 minutes.
H. The pH and the % solids are recorded. The pH is adjusted to fall in the range 7.2-7.4,
I. The entire product is then homogenized in two stages using a piston-type homogenizer at 180/30 bar (2500/500 psi) and passed through a UHT process at 144° C. (292° F.) for 5 seconds.
J. The beverage is collected in cans at 21-32° C. (70-90° F.), leaving a 12 mm (0.5 inches) headspace in the can. The product is then retorted at 121° C. (250° F.) for 7 minutes.
K. The result is a nutritional supplement that supplies about 20% of its energy as a soy protein hydrolysate.

### Example 17: Food Bar

The following example relates to a food bar that comprises a protein hydrolysate of the invention.

Formula:

| | Ingredient | Quantity (g) |
|---|---|---|
| Syrup | Corn Syrup, 63 D.E | 390.50 |
| | High Fructose Corn Syrup | 319.50 |
| | Glycerine | 566.00 |
| | Chocolate Flavour | 8.20 |
| | Vanilla Flavour | 2.00 |
| | Sub-total | 1286.20 |
| Powder | Supro ® Isolated Soy Protein | 300.00 |
| Blend | Soy Protein Hydrolysate | 300.00 |
| | Rice Syrup Solids | 32.40 |
| | Cocoa powder 12% fat | 76.40 |
| | Vitamin and Mineral premix | 10.50 |
| | Salt | 1.60 |
| | Sub-total | 720.90 |
| | Batch total | 2007.10 |

Process:
A. The isolated soy protein, soy protein hydrolysate, rice syrup solids (available from Natural Products, Lathrop, Calif.), cocoa powder (available from DeZaan, Milwaukee, Wis.), vitamin & mineral premix (available from Fortitech®, Schenectady, N.Y.), and 1.6 grams salt are added to a Winkworth mixer (available from Winkworth Machinery, Ltd., Reading, England) mixing at a speed of 48 revolutions per minute (rpm) for one minute.
B. In a separate container, a second mixture containing the liquid sugar syrups, the glycerine and the liquid flavoring agents is heated to a temperature of 37.8° C. (100° F.) by microwaving on high power for about 45 seconds. The liquid sugar syrup consists of a 55:45 blend of 63 DE corn syrup (available from Roquette®, LESTREM Cedex, France) and high fructose corn syrup 55 (available from International Molasses Corp., Rochelle Park, N.J.) and 566.0 grams glycerine. The liquid flavoring agents consist of 4.1 grams Edlong® Chocolate flavor 610 (available from The Edlong® Corporation, Elk Grove Village, Ill.), 4.1 grams Edlong® Chocolate flavor 614 (available from The Edlong® Corporation, Elk Grove Village, Ill.), and vanilla flavoring (available from Sethness Greenleaf, Inc., Chicago, Ill.). This heated second mixture is then mixed into the first mixture in a Winkworth mixer at a speed of 48 rpm for 3 minutes and forty-five seconds. The resulting dough is then sheeted out onto a marble slab and bars are cut into pieces weighing from about 45 grams to about 55 grams (the bar pieces are 102 millimetres in length, 10 millimetres in height, and 35 millimetres wide).

### Example 18: Protein Extrudates

The following example relates to an extruded, protein enriched, expanded cereal that comprises a protein hydrolysate of the invention.

Dry Blend Formulations:

| | Formulations | | |
|---|---|---|---|
| Ingredients: | A (kg) | B (kg) | C (kg) |
| Soy Protein Hydrolysate | 50.0 | 85.0 | 99.0 |
| Rice Flour | 49.0 | 14.0 | 0.0 |
| Calcium Carbonate | 0.7 | 0.7 | 0.7 |
| Salt, fine grind | 0.3 | 0.3 | 0.3 |

### I. Mixing of Ingredients:

A. The ingredients of the Dry Blend Formulation are weighed and added into a ribbon blender.
B. The Dry Blend Formulation is then mixed until ingredients are uniformly distributed creating an admixture.
C. The admixture is then transferred to a hopper, where it is held for delivery via screw feeder to a pre-conditioner.

### II. Extrusion of Ingredients:

A. The admixture delivered to the pre-conditioner may be mixed with water or steam to produce a conditioned feed mixture.
B. The amount of water or steam added to create the conditioned feed mixture is dependent on the desired output from the extruder.
C. The conditioned feed mixture is then transferred into a Wenger TX-52 Mag extruder.
D. The transferred conditioned feed mixture is heated by the mechanical pressure and shear generated from the configuration of screw elements within the extruder forming a molten extrusion mass.
E. The extrudate exits the extruder through a die to give it shape.
F. The extrudate undergoes expansion due to water vapor flash upon leaving the die.
G. The expanded extrudate is cut to the desired length using a rotary knife or other type of cutting apparatus.

### III. Drying Process

A. The expanded, cut extrudate is transferred to a single passed, single zone, steam heated Proctor dryer.
B. The extrudate is dried to a moisture or water activity level providing the desired texture and microbial stability.
C. The drying process may include a toasting step to darken the color or to add flavor to the extruded piece.

Processing Conditions:

| Extrusion Parameters | | Values |
|---|---|---|
| Dry Formula Feed Rate | (kg/hr) | 50-80 |
| Cylinder Steam | (kg/hr) | 3.0-15.0 |
| Cylinder Water | (kg/hr) | 3.0-10.0 |
| Extruder Water | (kg/hr) | 6.0-30.0 |
| Cylinder Paddle Speed | RPM | 250-300 |
| Extruder Screw Speed | RPM | 250-450 |
| Knife Speed | RPM | 2000-2400 |
| SME (Specific Mech. Energy) | kWh/hr | 45-125 |
| Down Spout Temperature | (° C.) | 40-85 |
| Zone #1 Temperature | (° C.) | 35-55 |
| Zone #2 Temperature | (° C.) | 40-85 |
| Zone #3 Temperature | (° C.) | 80-120 |
| Zone #4 Temperature | (° C.) | 80-120 |
| Head Pressure | (PSIG) | 300-850 |
| Dryer Information | | Proctor Single Pass |
| Dryer Belt Speed Setting | | 4-12 |
| Temperature of the Dryer- Zone 1 | (° C.) | 105-200 |
| Time in the Dryer | (min) | 10-20 |
| Die Information | | |
| Spacer | | 6.35 mm (0.25 in.) |
| Insert: | | 1 mm × 4 mm, 4 slots |
| Cutter Information | | 6 blades, rotary |

The produced expanded soy protein extrudate's dry bulk density ranges from 0.10 g/mL to 0.70 g/mL.

The admixture or the extrudate may have inclusion of flavorings, colorants, seasonings, or nutrition adding ingredients by means well known to the art.

These extruded and expanded pieces may be coated with flavorings, colorants, seasonings, or nutrition adding ingredients by means well known to the art.

### Example 19: Acid Beverage

The following example relates to an acid beverage that comprises a protein hydrolysate of the invention.

Formula:

| Ingredient | g | % |
|---|---|---|
| Hydrolysed soy protein | 372 | 3.72 |
| High Fructose Corn Syrup | 1180 | 11.8 |
| Apple Juice Concentrate (68 Brix) | 131 | 1.31 |
| Citric Acid, Anhydrous | 20 | 0.2 |
| Water | 8297 | 82.97 |
| Total | 10000 | 100 |

In this example, hydrolyzed soy protein is fractionated at acid pH to separate insoluble from soluble peptides, thus creating acid soluble peptides for use in an acid beverage. For example, hydrolyzed and spray-dried soy protein is resuspended at a solids concentration of 2% in 40 L of deionized water using an overhead mixer, and the pH adjusted to 3.0 with HCl. Mixing is continued for 15 minutes at ambient temperature (about 22° C.) to ensure adequate hydration of the sample. The suspension is next centrifuged at 500×g to remove the bulk of the insoluble material. The supernatant is introduced into an OPTISEP 3000 filtration module containing a regenerated cellulose (RC) ultrafiltration membrane having a pore size of about 10 kDa. Passage of the supernatant through the ultrafiltration membrane forms a permeate containing acid soluble peptides with a MW of less than about 10 kDa, and a retentate containing aggregated (insoluble) peptides.

Process:
A. The soluble fraction of the soy protein hydrolysate is added to deionized water in a suitable mixing vessel and agitated with moderate shear. Food grade antifoam is added, if required, to control foam formation.
B. Mixing continues until the soy protein hydrolysate is evenly dispersed. The dispersion is then heated to 74°-79° C. (165-175° F.) and mixed for an additional 10 minutes.
C. The high fructose corn syrup, apple juice concentrate (68 Brix), and anhydrous citric acid are then added with continuous mixing.
D. The pH is adjusted to fall within the range 3.8-4.0 with an 85% citric acid solution.
E. The contents are homogenized at 180 bar (2500 psi), single stage using a high pressure, piston homogenizer.
F. The product is then pasteurized at 107° C. for 7 seconds.
G. The product is filled, hot, into sterile bottles and then placed in an ice bath to bring the temperature of the beverage to about room temperature. The bottled product is then stored in a refrigerator at ±5° C.

The result is an acid, ready-to-drink beverage in which about 25% of calories are provided by a soy protein hydrolysate.

### Example 20: Enriched Fruit Juice

The following example relates to an enriched fruit juice that comprises a protein hydrolysate of the invention.

Formula:

| Ingredient | % | solids basis % |
|---|---|---|
| Orange juice, concentrate x ~5 (63.2 Brix) | 17 | 10.744 |
| Soy Protein Hydrolysate | 2.86 | 2.5168 |
| Deionised Water | 80.14 | 0 |
| Total | 100 | 13.2608 |

Process:
A. Concentrated orange juice is added to a suitable mixing vessel.
B. Deionized water at 20-25° C. is added to a second container.
C. The soy protein hydrolysate is added, and a slurry is formed by mixing with moderate agitation. Food grade antifoam is used as necessary to control the foam that may form at this stage.
D. The soy protein hydrolysate slurry is heated to 70-80° C. and then homogenized at 200 bar (2800 psi).
E. The homogenized soy protein hydrolysate slurry is added to the concentrated orange with moderate shear mixing to produce a soy protein hydrolysate-fortified orange juice. Agitation continues for 5 minutes, or until the mixture appears homogeneous.
F. At this point the pH is adjusted, if necessary, into the range 3.5-3.7 using citric acid or potassium hydroxide.
G. The fortified orange juice is heated again to 70-80° C. and homogenized at 200 bar (2800 psi).
H. The product is then pasteurized at 90° C. and filled hot into sterile containers, then stored in ice water to reduce its temperature to <5° C.

The result is a soy protein hydrolysate-fortified orange juice, in which about 20% energy is derived from the soy protein hydrolysate.

### Example 21: Enriched Pasta

The following example relates to a pasta (˜360 Kcal/100 g) that comprises a soy protein hydrolysate of the invention.

Pasta is formed according to typical industry processing techniques using a pasta press following the step-by-step process below. The following table is a list of ingredients and the amounts used in percent of formulation.

| | | Lower Level | Higher Level |
|---|---|---|---|
| | Control | Hydrolysate | Hydrolysate |
| Ingredients | (%) | (%) | (%) |
| Durum Semolina | 100 | 90 | 77.2 |
| Soy Protein Hydrolysate | 0 | 10 | 20 |
| Vital Wheat Gluten | 0 | 0 | 2.8 |

The ingredients are combined and processed according to the following steps to produce the pasta:

### I. Production of Control

A. The durum semolina is combined with water in a mixing chamber before the extruder. Sufficient water is added to bring the moisture of the product exiting the press to about 30 to 32% moisture (as-is basis). The hydrated semolina is extruded through a die and cut to length.
B. Product is dried using a typical high temperature drying cycle for pasta.

### II. Production of Lower Level Hydrolysate

A. The dry ingredients are blended using a V blender.
B. The dry ingredient blend is combined with water in a mixing chamber before the extruder. Sufficient water is added to have the pasta press run at about the same motor load as the Control product. The hydrated blend is extruded through a die and cut to length.
C. Product is dried using a typical high temperature drying cycle for pasta.

### III. Production of Higher Level Hydrolysate

A. The dry ingredients are blended using a V blender.
B. The dry ingredient blend is combined with water in a mixing chamber before the extruder. Sufficient water is added to have the pasta press run at about the same motor load as the Control product. The hydrated blend is extruded through a die and cut to length.
C. Product is dried using a typical high temperature drying cycle for pasta.

The result is a pasta that has an increased quantity of soy protein hydrolysate at about 10% (lower level hydrolysate) to about 20% (higher level hydrolystate) total energy, on a packaged basis.

### Example 22: Ham

The following example relates to a ham that comprises a protein hydrolysate of the invention.

Formula:

| | Injection % | 60 | Extension % | 60.000 |
|---|---|---|---|---|
| | BRINE | | GREEN WEIGHT | |
| Ingredients | Kg | % | Kg | % |
| Deboned Ham | 100 | g | 62.499 | 62.499 |
| Water | 63.996 | 63.996 | 27.386 | 27.386 |
| Soy Protein Hydrolysate | 20 | 20 | 7.5 | 7.500 |
| Salt | 2.98 | 2.98 | 1.117 | 1.117 |
| Dextrose | 7.152 | 7.152 | 2.682 | 2.682 |
| Corn Syrup Solids | 4.768 | 4.768 | 1.788 | 1.788 |
| Sodium | 0.744 | 0.744 | 0.279 | 0.279 |
| Tripolyphosphate | | | | |
| Salt, Prague Powder | 0.286 | 0.286 | 0.107 | 0.107 |
| Erythorbate | 0.074 | 0.074 | 0.028 | 0.028 |
| Total | 100 | 100 | 103.39 | 100.00 |

Brine Preparation

Phosphates are dissolved in cold water making sure complete dispersion is effected for proper functionality to be achieved.

Salt and cure salt are then added and mixed until completely dissolved.

Soy protein hydrolysate and seasoning are added and mixed until evenly suspended.

Cure accelerators (sodium erythorbate or sodium ascorbate) are added last to the brine in order to prevent nitrite from converting to nitrous oxide gas.

The brine is agitated before and during injection to optimize suspension of the ingredients. Ice or chilled water is used to maintain brine temperature less than −12° C. (−11 to −17° C. is the optimum temperature).

Injection and Tumbling Procedures

The pork muscle is trimmed to remove excess fat and connective tissue.

A multi-needle injector is used to incorporate the brine solution into the pork muscles. Multiple passes through the injector may be required to achieve the targeted pump level and proper brine distribution within the pork muscles.

The pork muscles are macerated at a depth of 6-12 mm (0.25 to 0.5 inches) to increase surface area of muscle pieces. Proper maceration increases brine absorption and protein extraction necessary for binding muscle pieces together. Deeper maceration is required if the injection step is omitted for uniform distribution of brine ingredients within the muscle tissue.

The injected macerated muscle pieces are tumbled in a vacuum tumbler (Inject Star Tumbler) until brine pickup is complete and salt soluble proteins have been extracted to muscle surface. If desired, finely ground lean ham trimmings may be added at a maximum of about 15% of the total fresh meat weight during the tumbling process, along with the necessary brine solution to cure trimmings. The tumbled product may be held refrigerated for 12 hours before stuffing to optimize cook yields.

The tumbled product is then stuffed into casings and heat processed to a minimum 64.4° C. internal temperature.

After chilling, the product is vacuum packaged and refrigerated.

The result is a ham that has an level of soy protein hydrolysate contributing about 12.25% calories, but that retain the taste, aroma, structure, and mouthfeel of typical fresh hams currently on the market.

### Example 23: Enriched Flaked Cereal

The following example relates to a protein enriched extruded flaked cereal (PEEFC; ˜320 Kcal/100 g) that comprises a protein hydrolysate of the invention.

An extruded flaked cereal is formed according to typical industry processing techniques using a twin-screw cooking extruder with a barrel vent following the process below.

| | Control | PEEFC Formulas | | | |
|---|---|---|---|---|---|
| Ingredients | (kg) | A (kg) | B (kg) | C (kg) | D (kg) |
| Corn Flour | 92.2 | 82.2 | 64.5 | 44.5 | 0.0 |
| Sugar | 6 | 6 | 6 | 6 | 0.7 |
| Salt | 1.8 | 1.8 | 0.5 | 0.5 | 0.3 |
| Soy Protein Hydrolysate | 0 | 11 | 30 | 50 | 99 |

Dry blend formulations:

### I. Mixing of Ingredients:

A. The ingredients of the Dry Blend Formulation are weighed and added into a ribbon blender.
B. The Dry Blend Formulation is then mixed until ingredients are uniformly distributed creating an admixture.
C. The admixture is then transferred to a hopper, where it is held for delivery via screw feeder to a pre-conditioner.

### II. Extrusion of Ingredients:

A. The admixture delivered to the pre-conditioner may be mixed with water or steam to produce a conditioned feed mixture.
B. The amount of water or steam added to create the conditioned feed mixture is dependent on the desired output from the extruder.
C. The conditioned feed mixture is then transferred into a Wenger TX-52 Mag extruder with a vent port.
D. The transferred conditioned feed mixture is heated by the mechanical pressure and shear generated from the configuration of screw elements within the extruder forming a molten extrusion mass.
E. The extrudate is vented while contained within the extruder's barrel.
F. The post vented extrudate continues transversing the extruder through a cooling zone to densify and cool the extrudate.
G. The extrudate exits the extruder through a circular die opening to give it shape.
H. The shaped extrudate is cut to the desired length using a rotary knife or other type of cutting apparatus.
I. The resulting pellets are tempered then passed though flaking rolls to produce a desired flake thickness.

### III. Drying Process

A. The flaked extrudate is transferred to a single passed, single zone, steam heated Proctor dryer.
B. The resulting flakes are dried or toasted to give the desired moisture and appearance.
C. The extrudate is dried to a moisture or water activity level providing the desired texture and microbial stability.

Processing Conditions:

| Extrusion Parameters | | Values |
|---|---|---|
| Dry Formula Feed Rate | (kg/hr) | 15-30 |
| Cylinder Steam | (kg/hr) | 2.0-10.0 |
| Cylinder Water | (kg/hr) | 3.0-8.0 |
| Extruder Steam | (kg/hr) | 0.9-2.0 |
| Extruder Water | (kg/hr) | 6.0-30.0 |
| Cylinder Paddle Speed | (RPM) | 250-300 |
| Extruder Screw Speed | (RPM) | 250-450 |
| Knife Speed | (RPM) | 1000-2000 |
| SME (Specific Mech. Energy) | (kWh/hr) | 30-70 |
| Down Spout Temperature | (° C.) | 40-85 |
| Zone #1 Temperature | (° C.) | 35-75 |
| Zone #2 Temperature | (° C.) | 70-120 |
| Zone #3 Temperature | (° C.) | 80-120 |
| Zone #4 Temperature | (° C.) | 10-70 |
| Zone #5 Temperature | (° C.) | 10-70 |
| Head Pressure | (PSIG) | 0-50 |
| Flaking Rolls Information | | |
| Roll Gap | (mm) | 0.25-1.0 |
| Roll Speed | (RPM) | 10-50 |
| Roll Temperature | (° C.) | 20-40 |
| Dryer Information | | Proctor Single Pass |
| Temperature of the Dryer- Zone 1 | (° C.) | 95-105 |
| Time in the Dryer | (min) | 20-30 |

One skilled in the art would readily appreciate that the methods and compositions described herein are representative of exemplary embodiments, and not intended as limitations on the scope of the invention. It will be readily apparent to one skilled in the art that varying substitutions and modifications may be made to the present invention disclosed herein without departing from the spirit and scope of the invention. Thus, it should be understood that although the present disclosure has been specifically disclosed by embodiments and examples, modification and variation of the concepts herein disclosed may be resorted to by those skilled in the art, and that such modifications and variations are considered to be within the scope of this invention as defined by the appended claims.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

1. A protein hydrolysate composition comprising a mixture of polypeptide fragments, wherein the protein hydrolysate composition stimulates cholecystokinin (CCK) and glugacon-like peptide-1 (GLP-1) releasing activity.

2. The protein hydrolysate composition of claim 1, wherein about 30% to about 50% of polypeptides in the soluble fraction of the protein hydrolysate composition have a molecular weight greater than about 20 kDa.

3. The protein hydrolysate composition of claim 1, wherein the protein hydrolysate composition contains a soluble fraction, an insoluble fraction, or combinations thereof.

4. The protein hydrolysate composition of claim 1, wherein the protein hydrolysate composition consists essentially of a soluble fraction.

5. The protein hydrolysate composition of claim 1, wherein the protein hydrolysate composition is a product of hydrolysis of a plant protein material, an animal protein material, or combinations thereof.

6. The protein hydrolysate composition of claim 1, wherein the protein hydrolysate composition is a product of hydrolysis of a plant protein material and wherein the plant protein material is a leguminous or non-leguminous plant.

7. The protein hydrolysate composition of claim 6, wherein the plant protein material is derived from soy.

8. The protein hydrolysate composition of claim 7, wherein the plant protein material is a soy protein isolate, a soy protein concentrate, a soy flour, or combinations thereof.

9. The protein hydrolysate composition of claim 8, wherein the plant protein material is a soy protein isolate,

10. The protein hydrolysate composition of claim 1, wherein the protein hydrolysate composition stimulates CCK and GLP-1 releasing activity after digestion with pepsin and pancreatin.

11. A food product containing a protein hydrolysate composition of claim 1.

12. (canceled)

13. (canceled)

14. (canceled)

15. (canceled)

16. (canceled)

17. (canceled)

18. (canceled)

19. (canceled)

20. (canceled)

21. A method of inducing satiety comprising ingesting a protein hydrolysate composition of claim 1.

22. (canceled)

23. (canceled)

24. (canceled)

25. (canceled)

26. (canceled)

27. (canceled)

28. (canceled)

29. (canceled)

30. (canceled)

31. A method of inducing satiety comprising ingesting a food product containing a protein hydrolysate composition of claim 1.

32. (canceled)

33. (canceled)

34. (canceled)

35. (canceled)

36. (canceled)

37. (canceled)

38. (canceled)

39. (canceled)

40. (canceled)
